(19) United States
(12) Patent Application Publication
(10) Pub. No.: US 2003/0003582 A1
(43) Pub. Date: Jan. 2, 2003

(54) Trans-viral vector mediated gene transfer to the retina

(75) Inventors: John Wakefield, Birmingham, AL (US); Jean Bennett, Bryn Mawr, PA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Tranzyme, Inc.

(21) Appl. No.: 10/140,227

(22) Filed: May 7, 2002

Related U.S. Application Data
(60) Provisional application No. 60/340,116, filed on Nov. 2, 2001.
(60) Provisional application No. 60/289,459, filed on May 8, 2001.

(51) Int. Cl. A61K 48/00; C12N 15/867

(52) U.S. Cl. 435/456; 424/93.2

(57) ABSTRACT

Methods and compositions for the delivery and expression of a nucleotide sequence of interest to a cell of a mammalian eye, more particularly to a cell of the retina, more particularly to the retinal pigment epithelium (RPE) are provided. The methods and compositions of the present invention find use in modulating the expression of a nucleotide sequence of interest in a cell of a mammalian eye using the trans-viral vector system. The methods and compositions of the present invention find further use in the treatment and/or prevention of ocular disorders, particularly retinal degenerative disorders. The methods comprise administering to a retinal cell of a mammal in need thereof, a therapeutically effective amount of a trans-viral vector particle having a proviral genome comprising a nucleotide sequence of interest whose expression will lessen the clinical symptoms of the retinal disorder being treated.

### CROSS REFERENCE TO RELEATED APPLICATIONS

This application claims the benefit of U.S. Provisional Application No. 60/340,116 filed Nov. 2, 2001 and U.S. Provisional Application No. 60/289,459 filed May 8, 2001, both of which are herein incorporated in their entirety.

### FIELD OF THE INVENTION

The present invention is directed to a method for the delivery and expression of a nucleotide sequence of interest to a cell of a mammalian eye, more particularly to a cell of the retina.

### BACKGROUND OF THE INVENTION

A more detailed understanding of the molecular bases of various forms of inherited retinal degeneration has led to the recent development of promising gene therapy techniques for the treatment of these blinding diseases. Retinitis pigmentosa (RP), the most common inherited retinal degenerative disease, is a clinically and genetically heterogeneous group of ocular disorders affecting approximately 1 in 3000 people in all ethnic groups (Kaplan et al. (1990) Hum. Genet. 85:635). The existence of several naturally occurring and genetically engineered animal models has facilitated the study of gene therapy techniques for RP. Because systemic administration of gene therapy agents does not result in gene delivery to the retina, surgical delivery of various vectors is required to study their efficacy and safety in vivo.

Proof-of-principle of gene therapy-mediated rescue of photoreceptor degeneration has been demonstrated using adenovirus, adeno-associated virus, and a gutted adenovirus (for example, Bennett et al. (1996) Nat. Med. 2:649; Jomary et al. (1997) Gene Ther. 4:683; and, Kumar-Singh et al (1998) Hum. Mol. Genet. 7:1893). More recently, gene transfer to retinal cells and gene therapy-mediated rescue of photoreceptor degeneration have been accomplished using an HIV-based lentiviral vector (Miyoshi et al. (1997) Proc. Natl. Acad. Sci. USA 94:10319 and Takahashi et al. (1999) J. Virol. 73:7812.) Even more recently, progress has been made in the development of lentivirus-based vectors that are capable of efficient gene transfer and include important safety design features. For example, a new class of HIV-based vectors have been introduced to split the gag-pol component of the packaging construct into two separate parts: one expresses Gag/Gag-Pro and the other expresses Pol (Reverse Transcripatase and Integrase) fused with Vpr (Vpr-RT-IN) (Liu et. al. (1997) J. Virol. 71:7701-7710; Wu et al. (1997) EMBO J. 16:5113-5122; and Wu et al. (1995) J. Virol. 69:3389-3398), (Wu et al. (2000) Mol. Therapy 2,47-55). This “trans-viral” vector design reduces the risk of generating replication competent retrovirus through genetic recombination, and enables in vitro monitoring of trans-viral stocks for the existence of recombinants containing functional gag-pol as a means to quality assure safety against generating RCR in vivo.

Additional methods and compositions for the effective delivery and expression of nucleotide sequences to mammalian eye cells are needed.

### SUMMARY OF THE INVENTION

Methods and compositions for the delivery and expression of a nucleotide sequence of interest to a cell of a mammalian eye, more particularly to a cell of the retina, more particularly to the retinal pigment epithelium (RPE) are provided. The methods and compositions of the present invention find use in modulating the expression of a nucleotide sequence of interest in a cell of a mammalian eye.

Compositions of the present invention include a trans-viral vector particle, including a trans-retroviral vector and a trans-lenti viral vector particle. The vector particle of the present invention has a modified proviral genome that contains a nucleotide sequence of interest having retinal therapeutic properties operably linked to a promoter active in a target cell. In specific embodiments of the invention, the nucleotide sequence of interest encodes, for example, an antisense nucleotide sequence, a ribozyme, or a polypeptide. In further embodiments, the polypeptide encoded by the nucleotide sequence of interest is a growth factor. Also provided are pharmaceutical compositions comprising the trans-viral vector particle of the present invention. In specific embodiments of the invention, the nucleotide sequence of interest having retinal therapeutic properties encodes RPE65 or a biologically active fragment or variant thereof.

The present invention further provides a packaging cell line having stably incorporated an env construct, a packaging construct, a trans-enzyme construct, and a gene transfer vector, where the gene transfer vector comprises a modified proviral genome and a nucleotide sequence of interest having retinal therapeutic properties operably linked to a promoter active in a target cell. In specific embodiments, the nucleotide sequence of interest having retinal therapeutic properties encodes RPE65 or a biologically variant or fragment thereof.

The present invention further provides a method for delivering a nucleotide sequence of interest to a retinal cell of a mammal. The method comprises administering to a retinal cell a trans-viral vector particle, including a trans-retro viral particle and a trans-lenti viral particle. In specific embodiments, the trans-viral vector particle comprises a modified proviral genome having the nucleotide sequence of interest operably linked to a promoter active in said retinal cell, where the nucleotide sequence of interest has retinal therapeutic properties. In specific methods of the invention, the nucleotide sequence of interest having retinal therapeutic properties encodes RPE65, or a biologically active variant or fragment thereof.

The methods and compositions of the present invention find further use in the treatment and/or prevention of ocular disorders. The present invention provides a method for treating and/or preventing retinal disorders, including, but not limited to, retinal degenerative disorders. In specific embodiments, the retinal degenerative disorder is Leber congenital amaurosis. The methods comprise administering to a retinal cell of a mammal in need thereof, a therapeutically effective amount of a trans-viral vector particle having a modified proviral genome comprising a nucleotide sequence of interest whose expression will lessen the clinical symptoms of the retinal disorder being treated. The methods of the invention therefore find use in improving the clinical outcome of a mammal suffering from retinal disorders. In specific methods of the invention, the trans-viral particle comprises a nucleotide sequence of interest encoding RPE65 or a biologically active variant or fragment thereof and the viral particle is administered in a therapeutically effective amount for the treatment and/or prevention of Leber congenital amaurosis.

Further provided are methods for generating a trans-viral vector of the present invention. The method comprises (a) providing a retroviral packaging cell having an env construct, a packaging construct, and a trans-enzyme construct; (b) introducing into this packaging cell a gene transfer vector comprising a modified proviral genome comprising a nucleotide sequence of interest having retinal therapeutic properties operably linked to a promoter active in said cell; and, (c) incubating the packaging cell of step (b) under conditions wherein a viral vector particle is produced. In specific embodiments, the nucleotide sequence of interest having retinal therapeutic properties is RPE65 or a biologically active fragment or variant thereof.

### BRIEF DESCRIPTION OF THE DRAWINGS

FIG. 1 schematically illustrates a non-limiting example of a lentiviral vector that can be used in the methods of the present invention. Specifically, FIG. 1 provides an illustration of the “trans-lenti viral” vector design comprising four (4) different DNA constructs used to generate a trans-viral particle that can be used in the methods of the present invention. (Inactive Reverse Transcriptase and Integrase are unboxed.)

FIG. 2 provides a non-limited illustration of the trans-retroviral vector design.

FIG. 3 shows the percentage of injected mouse eyes possessing GFP plotted as a function of time (days) following subretinal delivery. Ophthalmoscopy was performed daily after injection of trans-lentiviral-GFP to monitor GFP expression by transduced retinal cells in vivo.

FIG. 4 shows an in vitro analysis of cell-mediated responses. Spleen-derived T-cells were collected from mice following injection with the trans-lentiviral-GFP. T-cell proliferation assays were used to characterize virus-specific (a), and GFP-specific (b) cytokine profiles 1 and 7 days following subretinal injection of trans-lentiviral-GFP. The same technique was performed to evaluate the lentivirus-specific (c) and GFP-specific (d) cytokine profiles following intramuscular injection of trans-lentivus-GFP. Individual cytokines are plotted on the x-axis, while the y-axis indicated the number of spots/well in the assay, corresponding directly to the number of T-cells elaborating a particular cytokine. Hatched bars indicate control assays: “−”=unstimulated spleen-derived T-cells (−control); +=Spleen-derived T-cells treated with the T-cell stimulant Phorbol 12-Myristate 13-Acetate (+control). *=p=0.001; **=p<0.000

FIG. 5 characterizes the humoral response. Serum samples were collected from mice before injection and 7 and 21 days after injection with trans-lentiviral-GFP. ELISA was used to identify trans-lentiviral-specific (a) and GFP-specific (b) antibody isotypes following subretinal administration of lentivirus-GFP. Isotyping of the trans-lentiviral-specific (c) and GFP-specific (d) humoral response was also studied in animals receiving intramuscular injection of trans-lentiviral-GFP. (*, p≦0.0001; Note the difference in scale in the y-axes of panels (c) and (d)).

### DETAILED DESCRIPTION OF THE INVENTION

The present invention is directed to compositions and methods for the delivery and expression of a nucleotide sequence of interest to a cell of a mammalian eye, more particularly to a cell of the retina, more particularly to the retinal pigment epithelium (RPE). In the methods of the present invention, the nucleotide sequence of interest is delivered to the cells of the mammalian retina in a viral vector particle.

The present invention demonstrates that administration of the “trans-viral” vector particle into the retina of a mammal results in rapid, efficient, and stable transduction of RPE cells. The recipient mammals demonstrated no antigen-specific delayed type hypersensitivity (DTH) and the analysis of cell-mediated responses in vitro revealed that subretinal administration of TLV induced antigen-specific Th2 type cytokines. Moreover, the humoral response was characterized by the induction of Th2 antibody isotypes, and by the absence of neutralizing antibodies. As discussed in more detail below, these findings have important implications with respect to therapeutic applications for the trans-viral vector mediated retinal gene transfer.

As used herein, “a viral vector particle” refers to a modified retrovirus having a modified proviral RNA genome which comprises a gene transfer vector containing a nucleotide sequence of interest operably linked to a promoter active in the target cell. The viral genome is packaged in a protein envelope that is compatible with, and capable of causing “infection” of a target cell. The infection of the viral vector particle results in the entry and subsequent insertion of the modified proviral viral genome (i.e., the retroviral transfer vector) into the genome of the target cell. As explained in more detail below, the viral vector particle used in the methods and compositions of the invention is a trans-viral vector that lacks the information necessary for the reconstitution of a complete viral particle in the target cell.

In one embodiment of the present invention, the trans-viral vector is used for the delivery of a nucleotide sequence of interest to a retinal cell of a mammal. In this manner, the level of a nucleotide sequence of interest in a retinal cell of a mammal is modulated (i.e. increased or decreased). A modulation in the level of the nucleotide sequence of interest comprises about a 1%, 5%, 10%, 20%, 40%, 80%, 100% or greater increase/decrease in the level of the nucleotide sequence in the retinal cell. In other embodiments, the modulation of the level of the nucleic acid sequence modulates the level/activity of a polypeptide in the target cell. In this instance, expression of the nucleotide sequence of interest results in about a 1%, 5%, 10%, 20%, 40%, 80%, 100%, or greater increase/decrease in the activity of a polypeptide in the retinal cell.

In another embodiment of the present invention, the delivery and expression of the nucleotide sequence of interest in a mammalian retinal cell finds use in improving the clinical outcome of a mammal having a retinal disorder. In this embodiment, the method comprises administering a therapeutically effective amount of a trans-viral vector particle comprising a nucleotide sequence of interest to retinal cells of a mammal. In this embodiment, the nucleotide sequence of interest has retinal therapeutic properties. While any retinal disorder (either inherited or non-inherited) may be treated or prevented by the methods of the present invention, of particular interest are retinal diseases or disorders that result in the degeneration of the retina. Such retinal degeneration is characterized by a loss of visual receptors (i.e., rods and cones) and is often accompanied by focal proliferation of the adjacent retinal pigment epithelium and migration of such cells into the sensory retina, where they appear as pigmented interstitial cells.

A variety of retinal degeneration ocular disorders are known in the art. See, Isselbacher et al. (1994) Harrison 's Principles of Internal Medicine, McGraw Hill, p. 102-104, which is herein incorporated by reference. For example, retinitis pigmentosa refers to one group of pigmentary retinopathies that is hereditary, bilateral, and progressive. The genetic basis of many of the retinitis pigmentosa (RP) disorders have been identified, and have lead to the genetic categorization of the RP disorders (i.e., autosomal dominant (adRP), autosomal recessive (arRP), x-linked (xIRP) or syndromic). In addition, RP disorders have been classified clinically into two broad categories including Type 1 (characterized by rapid progression and diffuse, severe pigmentation) and Type 2 (characterized by a slower progression and more regional and less severe pigmentation). For a review see, for example, Hauswirth et al. (2000) Investigative Ophthalmology & Visual Science 41:2821-2826, Bennett et al. (2000) Curr. Opin. Mol. Ther. 2:420-5, and Bennett et al. (2000) Mol. Ther. 1:501-505; all of which are herein incorporated by reference.

Additional retinal degenerative disorders include age-related macular degeneration, Leber congenital amaurosis, gyrate atrophy, Norrie disease, choroideremia, cone-rod dystrophy, Usher's syndrome, Sorsby's fundus dystrophy, Stargardt disease, Best dystrophy and North Carolina Macular dystrophy. In addition, there are numerous inherited systemic diseases such as abetalipoproteinemia, mucopolysaccharidosis VI, Bardet-Biedle, Charcot-Marie-Tooth, Batten disease and Refsum disease that include retinal degeneration among the symptoms. There are also retinal diseases resulting from abnormal expression of retina specific genes that are required for retinal development/differentiation. Examples of such diseases include Ocular Albinism, and some forms of cone-rod dystrophy.

Accordingly, the method of the invention “prevents” (i.e., delays or inhibits) and/or “reduces” (i.e., decreases, slows, or ameliorates) the detrimental effects of a retinal disease, injury, or disorder in the mammal receiving the nucleotide sequence of interest via the viral vector. The present invention therefore finds use in the treatment or prevention of an ocular disorder of the outer retina, such as retinitis pigmentosa, wherein the treatment or prevention comprises any enhanced improvement in either the rate or the extent of behavioral recovery of the mammal or an improvement in the morphological and/or electrophysiological preservation of the retinal photoreceptors.

I. Nucleotide Sequence of Interest

The present invention provides a method to express a nucleotide sequence of interest in a target cell (i.e., a retinal cell of a mammal, particularly, an RPE cell). The target cell can be from any animal, particularly the animal is a vertebrate, and more particularly the vertebrate is a mammal. In specific embodiments, the vertebrate is a human. In other embodiments the vertebrate is a non-human vertebrate. Such non-human vertebrates include, but are not limited to, rodents, non-human primates, ovines, bovines, ruminants, lagomorphs, porcines, caprines, equines, murines, canines, felines, aves, etc.

The nucleotide sequence of interest used in the compositions and methods of the present invention can be from any animal species including, but not limited to, rodents, non-human primates, ovines, bovines, ruminants, lagomorphs, porcines, caprines, equines, murines, canines, felines, aves, and humans. Additionally, the sequence may be a variant of a naturally occurring sequence or even a synthetic sequence. Preferably the nucleotide sequence administered is from the same species as the retinal cells being infected with the viral particle or has been modified to reflect the sequence of the recipient.

While it is recognized that a variety of nucleotide sequences of interest may be used in the methods of the present invention (i.e., molecular markers), of particular interest are nucleotide sequences having retinal therapeutic properties. By “a nucleotide sequence having retinal therapeutic properties” is intended a sequence which upon expression in the target cell prevents or reduces the detrimental effects of the retinal degenerative disorder (i.e., expression in the mammalian retinal cell will result in an improvement in either the morphological and/or electrophysiological preservation of the photoreceptor/RPE cells either in vivo or in vitro and/or the rate or the extent of behavioral recovery of the mammal suffering from a retinal degenerative disorder). It is recognized that a nucleotide sequence having retinal therapeutic properties may act on various levels (i.e., improving a specific morphological condition, improving a response to an agent, etc.). As discussed further below, the sequence may encode a polypeptide having beneficial therapeutic properties or alternatively, the sequence may encode a ribozyme or an antisense sequence that alters the expression and/or levels of an endogenous RNA transcript.

For instance, a nucleotide sequence of interest used in the methods and compositions of the present invention may comprise a nucleotide sequence encoding a polypeptide. Such a sequence may be native to the target cell (i.e., naturally expressed in the cell) or the nucleotide sequence may be heterologous or foreign to the target cell. In one embodiment, the nucleotide sequences are designed to compensate for lack-of-function mutations that result in retinal degenerative disorders (i.e., genes responsible for retinitis pigmentosa, macular degeneration, etc.). Such mutations are known in the art. Table 1 lists examples of such sequences. See, also, for example, Ret Net, The Retinal Information Network (www.sph.ath.tmc.edu/RetNet/ and Daiger et al. (1996) Invest Ophthalmol. U.S. Sci. 39:S295, both of which are herein incorporated by reference. Such a gene-augmentation approach has proven successful. For instance, expression of the nucleotide sequence encoding the beta subunit of rod-specific cGMP phosphodiesterase (PDEβ) has been shown to successfully rescue the null PDEβ phenotype (Bennett et al. (1996) Nat Med. 2:649-654; Kumar-Singh et al. (1998) Hum Mol Genet. 7:1893-1900 and Takahashi et al. (1999) J. Virology 73:7812-7816, all of which are herein incorporated by reference).

TABLE 1
Summary of Some Genes Involved in Retinal Disorders

| | Mapped Genes | Mapped and Cloned |
|---|---|---|
| Disease Category | (not Cloned) | Genes |
| Bardet-Biedl syndrome, autosomal recessive | BBS1, BBS3, BBS5 | BBS2, BBS4, MKKS |
| Chorioretinal atrophy or degeneration, autosomal | AA, MCDR1 | RGR |
| dominant | | |
| Cone or cone-rod dystrophy, autosomal dominant | CORD4, CORD5, CORD7, | CRX, GUCA1A, |
| | RCD1 | GUCY2D, UNC119 |
| Cone or cone-rod dystrophy, autosomal recessive | CORD5, CORD8, CORD9 | ABCA4, RDH5 |
| Cone or cone-rod dystrophy, X-linked | COD1, COD2 | |
| Congenital stationary night blindness, autosomal | | GNAT1, PDE6B, RHO |
| dominant | | |
| Congenital stationary night blindness, autosomal | | RDH5, RHOK |
| recessive | | |
| Congenital stationary night blindness, X-linked | | CACNA1F, NYX, RPGR |
| Deafness alone or Usher syndrome, autosomal recessive | USH2B | CDH23, MYO7A, USH1C |
| Leber congenital amaurosis, autosomal dominant | | CRX |
| | | AIPL1, CRB1, CRX, |
| Leber congenital amaurosis, autosomal recessive | LCA3, LCA5 | GUCY2D, LRAT, RPE65, |
| | | RPGRIP1 |
| Macular degeneration, autosomal dominant | ARMD1, CYMD, MCDR1, | EFEMP1, ELOVL4, RDS, |
| | STGD4 | TIMP3, VMD2 |
| Macular degeneration, autosomal recessive | | ABCA4 |
| Ocular-retinal developmental disease, autosomal | WGN1 | |
| dominant | | |
| Optic atrophy, autosomal dominant | OPA4 | OPA1 |
| Optic atrophy, X-linked | OPA2 | TIMM8A |
| | | CRX, FSCN2, NRL, |
| Retinitis pigmentosa, autosomal dominant | RP9, RP10, RP17, RP18 | PRPC8, PRPF31, RDS, |
| | | RHO, ROM1, RP1 |
| | | ABCA4, CNGA1, CNGB1, |
| | | CRB1, LRAT, MERTK, |
| Retinitis pigmentosa, autosomal recessive | RP22, RP25, RP26, RP28, | NR2E3, PDE6A, PDE6B, |
| | RP29 | RGR, RHO, RLBP1, |
| | | RPE65, SAG, TULP1, |
| | | USH2A |
| Retinitis pigmentosa, X-linked | RP6, RP23, RP24 | RP2, RPGR |
| Syndromic or systemic retinopathy, autosomal dominant | CORD1 | PAX2, SCA7 |
| | | CLN3, MTP, PEX1, |
| Syndromic or systemic retinopathy, autosomal recessive | ALMS1, AXPC1, MRST | PHYH, RBP4, TTPA, |
| | OPA3, WFS2 | WFS1 |
| Syndromic or systemic retinopathy, X-linked | | TIMM8A |
| | | CDH23, MYO7A, |
| Usher syndrome, autosomal recessive | USH1A, USH1E, USH2B, | PCDH15, USH1C, |
| | USH2C | USH2A, USH3A |
| Other retinopathy, autosomal dominant | AA, CACD, EVR1, EVR3, | ABCC6, JAG1, OPN1SW, |
| | VRNI | RB1 |
| | | ABCC6, CNGA3, CNGB3, |
| Other retinopathy, autosomal recessive | ACHM1, BCD, RNANC | NR2E3, OAT, PROML1, |
| | | RLBP1, SAG |
| Other retinopathy, mitochondrial | | KSS, LHON, MTATP6, |
| | | MTTL1, MTTS2 |
| | | CHM, DMD, NDP, |
| Other retinopathy, X-linked | AIED, PRD | OPN1LW, OPN1MW, |
| | | PGK1, RS1 |

As used herein “biologically active” fragments or variants of sequences of a nucleotide sequence of interest useful in the methods of the invention (i.e., a sequence having retinal therapeutic properties) retains substantially the same function as the respective native sequence. Such fragments will comprise at least about 10, 15 contiguous nucleotides, at least about 20 contiguous nucleotides, at least about 24, 50, 60, 80, 100, 120, 140, 160, 180, 200, 220, 240, 260, 280, 300, 340, 360, 380, or up to the entire contiguous nucleotides of the nucleotide sequence of interest. Methods for obtaining such fragments are known in the art and are described in further detail elsewhere herein.

By “variant” is intended substantially similar sequences. Thus, for nucleotide sequences or amino acid sequences, variants include sequences that are functionally equivalent to the nucleotide sequence of interest. Variant nucleotide sequences also include synthetically derived nucleotide sequences that have been generated, for example, by site directed mutagenesis but which still retain the function of the native sequence. Generally, nucleotide sequence variants or amino acid sequence variants of the invention will have at least 70%, generally 80%, 85%, 90%, 91%, 92%, 93%, 94%, 95%, 96%, 97%, 98%, or 99% sequence identity to its respective native nucleotide sequence. As discussed elsewhere herein, variants of the nucleotide sequences can encode amino acid sequences that differ conservatively because of the degeneracy of the genetic code. Methods of determining sequence identity are also discussed elsewhere herein.

With respect to the amino acid sequences for the various full length or mature polypeptides, variants include those polypeptides that are derived from the native polypeptides by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Such variants may result from, for example, genetic polymorphism or from human manipulation. As discussed elsewhere herein, methods for such manipulations are generally known in the art. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr. A variant of a native nucleotide sequence or native polypeptide has substantial identity to the native sequence or native polypeptide. A variant may differ by as few as 1 to 10 amino acid residues, such as 6-10, as few as 5, as few as 4, 3, 2, or even 1 amino acid residue. A variant of a nucleotide sequence may differ by as low as 1 to 30 nucleotides, such as 6 to 20, as low as 5, as few as 4, 3, 2, or even 1 nucleotide residue.

Retinal degenerative diseases are characterized by progressive apoptotic death of photoreceptors. Consequently, a nucleotide sequence of interest having a retinal therapeutic property that is useful in the methods and compositions of the present invention may encode a growth factor. As used herein a “growth factor” refers to any polypeptide having a growth, proliferative, or trophic effect on a photoreceptor and/or RPE cell of the retina. Growth factors useful in the methods of the present invention include, but are not limited to, members of the neurotrophin family (i.e., nerve growth factor (NGF), brain-derived neurotrophic factor (BDNF), neurotrophin-3 (NT-3), and neurotrophin-4 (NT-4, also known as NT-4/5 or NT-5); fibroblast growth factors (FGFs, i.e., basic fibroblast growth factor); epidermal growth factor family (i.e., EGF, TGFα, amphiregulin, heparin-binding EGF-like growth factor (HB-EGF), batacelluin (BTC), and the neuregulin group); platelet-derived growth factor; insulin; insulin-like growth factors (i.e., IGF-I and IGF-2); ciliary neurotrophic factor (CNTF), glia cell line-derived neurotrophic factor family (GDNF) (i.e., GDNF and neurturin (NTN), persephin (PSP), and artemin (ART)); transforming growth factor β superfamily (i.e., subfamilies include TGFβ1, TGFβ2, TGFβ3, TGFβ4, TGFβ5, activin, inhibin, decapentaplegic); growth differentiation factors (GDF); glia-derived nexin; activity dependent neurotrophic factor (ADNF); glial growth factor (GGF); and the like. It is further recognized that any biologically active variant of these growth factors are also useful in the methods of the present invention.

The use of such growth factors for the treatment or prevention of various forms of retinal degenerative disorders is known in the art. For example, growth factors of particular interest for the treatment of retinal pigmontosa include CNTF (Liang et al. (2001) Mol Ther 3:241-8; Caffe et al. (2001) Invest Ophthalmol Vis Sci 42:275-82); FGF (bFGF and FGF2 (Akimoto et al. (1999) Invest Ophthalmol Vis Sci 40:273-279); GDNF (Frasson et al. (1999) Nat Med. 5:1183-1187); and PEDF (pigmented epithelium-derived factor) (Cayouette et al. (1999) Neurobiol Dis. 6:523-532). All of these references are herein incorporated by reference.

Moreover, because apoptosis appears to be the common death pathway for photoreceptors in RP animal models, nucleotide sequences of interest also include those that modulate the activity of polypeptides in the apoptotic pathway. Such nucleotide sequences may encode the anti-apoptotic polypeptides Bcl-2, c-Fos, or IAP (Inhibitor of Apoptosis) (Xu et al. (1999) J Neurosci. 19:5026-5033). Alternatively, the nucleotide sequences of interest having retinal therapeutic properties may disrupt the expression of the pro-apoptotic genes, such as p53.

In another embodiment of the present invention, the nucleotide sequence of interest having retinal therapeutic properties comprises a ribozyme. Ribozymes are enzymes comprised of ribonucleic acid (RNA) that conduct a variety of reactions involving RNA, including cleavage and ligation of polynucleotide strands. The specificity of ribozymes is determined by base pairing (hydrogen bonding) between the targeting domain of the ribozyme and the substrate RNA. Altering the nucleotide sequence of the targeting domain can modify this specificity. The catalytic domain of a ribozyme can also be changed in order to increase the activity or stability of the enzyme. Methods of designing ribozymes are known in the art. See, for example, U.S. Pat. Nos. 5,646,031; 5,646,020; and 5,639,655; all of which are herein incorporated by reference.

Ribozymes can be used according to the methods of the present invention to treat or prevent retinal degenerative disorders. For instance, ribozyme sequences may be used for the treatment of retinal degenerative diseases, such as the autosomal dominant retinitis pigmentosa (ADRP). Specifically, a ribozyme designed to discriminate and catalyze the destruction of mRNA carrying a histidine substitution at codon 23 (P23H) has been developed. Expression of either a hammerhead or hairpin ribozyme in a rat model markedly slows the rate of photoreceptor degeneration for at least 15 months. See, for example, La Vail et al. (2000) PNAS 97:11488-11493; Drenser et al. (1998) Invest. Ophthalmol. Visual Sci. 39:681-689; and U.S. Pat. No. 6,225,291. It is recognized that the skilled artisan can construct nucleotide sequences encoding ribozymes that destroy mutant RNA molecules associated with other human RP or other retinal diseases. See, for example, Daiger et al. (1995) Behavioral Brain Sci. 18:452-467 and U.S. Pat. No. 6,225,291; both of which are herein incorporated by reference.

Alternatively, the nucleotide sequence may comprise an antisense nucleotide sequence. In this embodiment, the nucleotide sequence of interest comprises a nucleotide having a sequence complementary to the targeted sequence of the retinal cell. The nucleic acid targeted by the antisence sequence may be either DNA or RNA. The targeted nucleotide sequence will comprise a cellular gene (or mRNA transcribed from the gene) whose expression is associated with a particular retinal degenerative disorder (i.e., a retinitis pigmentosa disorder). The site or sites within the targeted gene where the antisense interaction is to occur will be such as to modulate the expression of the polypeptide encoded by the targeted sequence (either at the level of transcription or translation), and thereby treat or prevent the retinal degenerative disorder. Preferred sites to target with the antisense oligonucleotide include the region encompassing the translation initiation or termination codon of the open reading frame (ORF) of the targeted gene. The transcription initiation site, or “5′ cap site” and the 5′ cap region (which encompasses from about 25 to about 50 contiguous nucleotides at the extreme 5′ terminus of a capped mRNA) may also be effective targets of the antisense nucleotide sequence.

Once the target site has been identified, oligonucleotides are chosen which are sufficiently complementary to the target sequence (i.e., hybridize with sufficient specificity to give the desired effect.) The term “specifically hybridize” refers to a sufficient degree of complementarity such that stable and specific binding occurs between the target and the oligonucleotide and thereby prevents transcription or translation of the target sequence. It is understood in the art that an oligonucleotide need not be 100% complementary to its target nucleic acid sequence to specifically hybridize to its target. It is further recognized that there must be a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under conditions in which the assays are conducted.

One of skill in the art will recognize that nucleotide sequences of interest having retinal therapeutic properties may be administered in various combinations to the retinal cells of the mammals to achieve the desired therapeutic effect. For example, anti-apoptotic therapy can be combined with gene augmentation to achieve an additive therapeutic effect. See, for example, Bennett et al (1998) Gene Ther 5:1156-1164.

One of the most clinically severe retinal degenerations is Leber congenital amaurosis (LCA). LCA causes near total blindness in infancy and can result from mutations in RPE65. Delivery of the gene encoding RPE65 to RPE cells in vivo has provided previously blind Briard dogs with vision (Acland et al. (2001) Nature Genetics 28: 92-5). In one embodiment of the present invention, the methods and compositions of the invention find use in the treatment of LCA. In this embodiment, the trans-viral particle comprises a nucleotide sequence of interest encoding RPE65 or a biologically active fragment or variant thereof.

The RPE65 sequence is known in the art. See, for example, Aguirre et al. (1998) Mol. Vis. 4:23 (canine sequence); Genbank Accession No. NM029987 and Simon et al. (1995) J. biol. Chem. 270:1107-1112 (Mus musculus); NM053526 and Manes et al. (1998) FEBS Lettt. 423:133-137 (Rattus Norvegicus); or Genbank Accession No. HSRPE65S10 and Nicoletti et al. (1995) Hum. Mol Genetics 4:641-649; (human); Hamel et al. (1993) J. Biol. Chem. 268:15751-15757 (bovine); all of which are herein incorporated by reference. A biologically active variant or fragment of a nucleotide sequence encoding an RPE65 polypeptide will retain the ability to delay retinal degeneration when expressed in retinal cells characterized as having an RPE65 deficiency. See Example 2 for further descriptions of assays to measure retinal degeneration.

II. The Trans-Viral Vector System

The methods and compositions of the present invention allow for the delivery of a nucleotide sequence of interest to a cell of a mammalian eye, more particularly, the retinal cell of a mammal. Specifically, the methods of the invention use the “trans-viral vector system” for the generation of trans-viral particles for the delivery of the nucleotide sequence of interest.

In one embodiment of the present invention, the lentiviral vector used comprises a “trans-viral” vector. As used herein, a lentiviral vector having a “trans-viral vector” design is characterized as separating, at least in part, nucleotide sequence encoding the Gag and the Pol polyproteins. By “polyprotein” is intended a single precursor polypeptide which is processed into individual proteins. For example, the HIV Pol polyprotein comprises Reverse Transcriptase and Integrase. The HIV Gag polyprotein comprises, for example, MA, CA, NC, and p6.

In the trans-viral vector design, the nucleotide sequence encoding the Gag-Pro-Pol polyprotein is split into at least two separate parts. The first DNA segment comprises the nucleotide sequence encoding Gag or Gag/Pro or functional equivalents thereof, and at least a second DNA segment that encodes Reverse Transcriptase and/or Integrase or function equivalents thereof. In other words, a trans-viral system is distinguishable from other viral vector systems in that the polypeptides encoding Reverse Transcriptase and Integrase are supplied in trans from at least one other DNA segment than the DNA segment encoding a functional Gag polypeptide. Consequently, the trans-viral vector system allows for a safer viral vector, in part, by diminishing the likelihood of generating replication competent retrovirus through genetic recombination.

In one embodiment, the trans vector design encompasses a “trans-lenti viral vector.” The “trans-lenti” viral vector design is characterized by expressing the Gag-Pro-Pol polyprotein in at least two parts: a first DNA segment that expresses Gag or Gag-Pro and at least a second DNA segment that expresses Reverse Transcriptase and/or Integrase. “Trans-lenti” viral vector design is further characterized by the use of a Vpr and/or Vpx polypeptide or a functional equivalent thereof to target the Reverse Transcriptase and Integrase to the viral particle. In this design, the Vpr and/or Vpx polypeptides are used as vehicles to deliver functional Reverse Transcriptase and Integrase into the viral particle. Further details regarding the design of the Vpr/Vpx fusion proteins used in the trans-lenti viral vector design are outlined below.

In yet another embodiment, the trans-vector design encompasses a “trans-retroviral vector” design. As explained in further detail below, the “trans-retroviral” vector is characterized by expressing the Gag-Pro-Pol polyprotein in at least two parts: a first DNA segment that expresses Gag or Gag-Pro and at least a second DNA segment that expresses Reverse Transcriptase and/or Integrase. The “trans-retroviral” vector design is further characterized by the use of at least a fragment of the Gag polypeptide that is capable of being targeted to the viral particle, or a functional equivalent thereof, to target the Reverse Transcriptase and Integrase to the viral particle. In this design, the fragment of the Gag polypeptide is used as vehicles to deliver functional Reverse Transcriptase and Integrase into the viral particle. Further details regarding the design of the trans-retroviral vector design are outlined below.

One of skill will recognize that the trans-retroviral vector design can be used in viral vectors derived from any retroviral source. Accordingly, in specific embodiments of the present invention, a trans-retroviral vector is derived from a retrovirus other than a lentivirus. Such retroviral vectors can be derived from, but not limited to, retrovirus, including but not limited to, Moloney Leukemia Virus (MLV), Abelson murine leukemia virus, AKR (endogenous) murine leukemia virus, Avian carcinoma, Mill Hill virus 2, Avian Leukosis virus—RSA, Avian myeloblastosis virus, Avian myelocytomatosis virus 29, Bovine syncytial virus, Caprine arthritis encephalitis virus, Chick syncytial virus, Equine infectious anemia virus, Feline leukemia virus, Feline syncytial virus, Finkel-Biskis-Jinkins murine sarcoma virus, Friend murine leukemia virus, Fujinami sarcoma virus, Gardner-Arnstein feline sarcoma virus, Gibbon ape leukemia virus, Guinea pig type C oncovirus, Hardy-Zuckerman feline sarcoma virus, Harvey murine sarcoma virus, Human foamy virus, Human spumavirus, Human T-lymphotropic virus 1, Human T-lymphototropic virus 2, Jaagsiekte virus, Kirsten murine sarcoma virus, Langur virus, Mason-Pfizer monkey virus, Moloney murine sarcoma virus, Mouse mammary tumor virus, Ovine pulmonary adenocarcinoma virus, Porcine type C oncovirus, Reticuloendotheliosis virus, Rous sarcoma virus, Simian foamy virus, Simian sarcoma virus, Simian T-lymphotropic virus, Simian type D virus 1, Snyder-Theilen feline sarcoma virus, Squirrel monkey retrovirus, Trager duck spleen necrosis virus, UR2 sarcoma virus, Viper retrovirus, Visna/maedi virus, Woolly monkey sarcoma virus, and Y73 sarcoma virus human-, simian-, feline-, and bovine immunodeficiency viruses (HIV, SIV, FIV, BIV). See also, U.S. patent application Ser. No. 09/578,548.

A complete description of the “trans” viral vector design, the “trans-lenti” viral vector design, and the “trans-retroviral” vector design and the viral vectors systems used to produce these viral particles has been described in detail in U.S. patent application Ser. Nos. 09/089,900; 09/709,751; 09/460,548; U.S. Pat. No. 6,001,985; PCT Patent Application No. PCT/US00/18597, in Wu et al. (1997) EMBO 16:5113-5122; all of which are herein incorporated by reference.

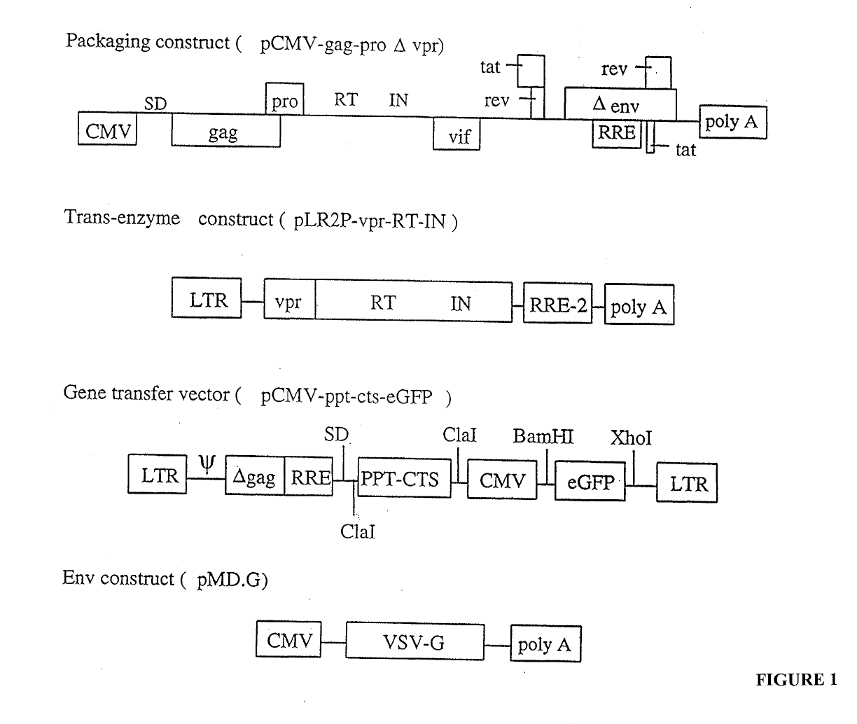

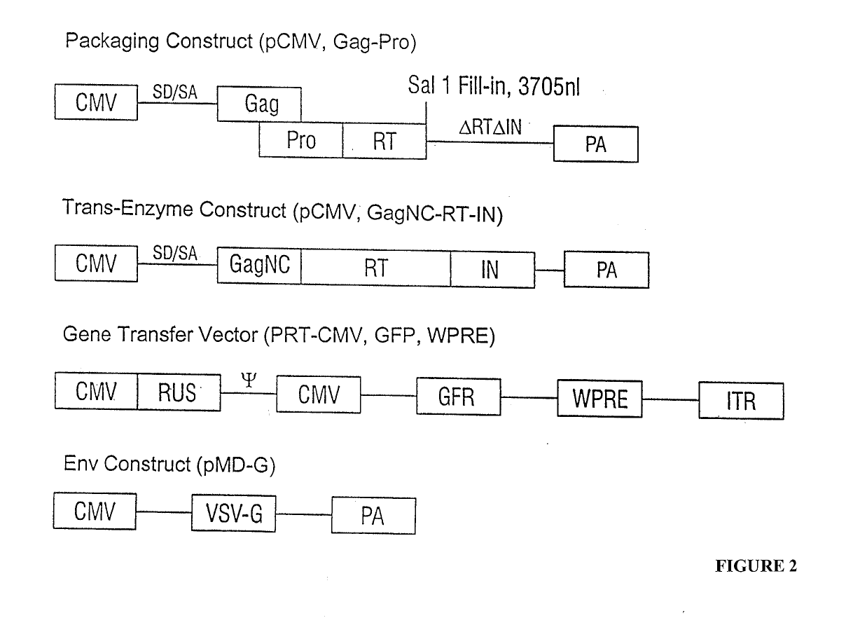

A non-limiting illustration of the trans viral vector system used in the methods of the present invention is provided in FIGS. 1-3. In these examples, the trans-vector system comprises the following components: an env construct, a packaging construct, a trans-enzyme construct, and a retroviral gene transfer vector. The “packaging construct” of the trans-viral system comprises a nucleotide sequence encoding Gag/Pro (represented as boxed structures in FIGS. 1-2, while the nucleotide sequences encoding Reverse Transcriptase (RT) and Integrase (IN) have been either deleted completely from the construct or disrupted in some manner that prevents the expression of a functional polypeptide. The nucleotide sequences encoding the Reverse Transcriptase and Integrase polypeptides are provided in trans to the packaging construct on a stretch of DNA referred to herein as the “trans-enzyme construct”. The viral expression system thereby disarms the Gag-Pro-Pol structure by splitting Gag-Pro from the nucleotide sequences encoding Reverse Transcriptase and Integrase.

The trans-viral vectors produced by the system of the present invention can be distinguished physically from lentiviral vectors that use a three vector system where the Gag/Pol is expressed as a single translation product. See, for example, Wu et al. (1997) EMBO J 16:5113-5122 and Wu et al. (2000) Mol. Therapy 1:47-55, which provide assays to identify uncleaved Vpr fusion proteins in the trans-lentivirus particles and assays that measure a reduced level of genetic recombination in the trans-lentiviral vector when compared to the three vector lentiviral vectors.

As used herein “nucleic acid sequences” will sometimes be used as a generic term encompassing both DNA and RNA fragments. As the materials of the invention include modified retroviral genomes and their proviral counterparts, particular functional sequences referred to will occur both in RNA and DNA form. The corresponding loci will be referred to interchangeably for their occurrences in both DNA and RNA. For example, the ψ packaging signal functions in the retroviral RNA genome as a packaging signal; however, the corresponding sequences occur in the proviral DNA. Similarly, promoter, enhancer, and terminator sequences occur, though in slightly different forms, in both the genomic RNA and proviral DNA forms. The interchangeability of these functionalities in the various phases of the viral life cycle is understood by those in the art, and accordingly, rather loose terminology in regard to DNA or RNA status is often used in referring to them. Specifically, sequences specified by a progression of bases should be understood to include these specific sequences and their complements, both in DNA and RNA forms.

While a description of the various elements contained on the gene transfer vector, the packaging construct, the envelope construct, and the trans-enzyme construct of the trans-viral vector system are provided below, it is recognized that one of skill in the art can readily generate “functionally equivalent” constructs. By “functionally equivalent” construct is intended each DNA construct (i.e., the packaging construct, the gene transfer vector, the envelope construct, and the trans-enzyme construct) have substantially the same function as the specific vector constructions illustrated herein. It is further recognized that the genetic elements in the various vectors of the trans-viral vector system may be from any viral source, particularly a retrovirus or a lentiviral source. Examples of such viral sources are provided elsewhere herein.

Examples and assays for the functional equivalence of the various components of the vector system used in the present invention are described more fully below. While Table 2 provides a reference for various genetic elements of the HIV-1 genome and is based on NCBI Genbank Accession Number AF033819, it is recognized that sequences from other lentiviruses are known in the art and can be used to construct functionally equivalent vectors and vector systems directed to a given host species of animal. A more detailed explanation of the components outlined in Table 1 and their function may be found in Coffin et al. (1997) Retroviruses, Cold Spring Harbor Laboratory Press, New York, herein incorporated by reference. Moreover, those of skill will appreciate that allelic variations in the various genetic elements exist between different isolates of the viruses and such variants may be used in the constructs of the present invention. For instance, such viral isolates are described in Li et al. (1992) J. Virol. 66:6587; Ghosh et al. (1993) Virology 194:858; and, U.S. Pat. No. 5,869,313; all of which are herein incorporated by reference.

TABLE 2
Genetic Elements and Coordinates of a Human HIV-1 Isolate

| Genetic Element | Coordinates |
|---|---|
| R: | (1-96) |
| U5: | (97-181) |
| PBS: | (182-199) |
| gag: | (336-1836) |
| pro: | (1637-2099) |
| pol: | (2102-4640) |
| vif: | (4587-5163) |
| vpr: | (5105-5339) |
| tat: | (5377-5591, 7925-7968) |
| rev: | (5516-5591, 7925-8197) |
| vpu: | (5608-5854) |
| env: | (5771-8339) |
| nef. | (8343-8710) |
| PPT: | (8615-8630) |
| U3: | (8631-9085) |
| R: | (9086-9181) |

Functionally equivalent sequences of the present invention also encompass various fragments of a retroviral genome that retain substantially the same function as the respective native sequence. Such fragments will comprise at least about 10, 15 contiguous nucleotides, at least about 20 contiguous nucleotides, at least about 24, 50, 60, 80, 100, 120, 140, 160, 180, 200, 220, 240, 260, 280, 300, 340, 360, 380, or up to the entire contiguous nucleotides of the specific genetic element of interest. Such fragments may be obtained by use of restriction enzymes to cleave the native viral genome; by synthesizing a nucleotide sequence from the native nucleotide sequence of the virus genome; or may be obtained through the use of PCR technology. See particularly Mullis et al. (1987) Methods Enzymol. 155:335-350, and Erlich, ed. (1989) PCR Technology (Stockton Press, New York). Again, variants of the various vector components, such as those resulting from site-directed mutagenesis, are encompassed by the methods of the present invention. As described in more detail below, methods are available in the art for determining functional equivalence.

By “variant” is intended substantially similar sequences. Thus, for nucleotide sequences or amino acid sequences, variants include sequences that are functionally equivalent to the various components of the viral vector system. Variant nucleotide sequences also include synthetically derived nucleotide sequences that have been generated, for example, by site directed mutagenesis but which still retain the function of the native sequence. Generally, nucleotide sequence variants or amino acid sequence variants of the invention will have at least 70%, generally 80%, 85%, 90%, 91%, 92%, 93%, 94%, 95%, 96%, 97%, 98%, or 99% sequence identity to its respective native nucleotide sequence.

Variants of the nucleotide sequences can encode amino acid sequences that differ conservatively because of the degeneracy of the genetic code. These naturally occurring allelic variants can be identified with the use of well-known molecular biology techniques, such as polymerase chain reaction (PCR) and hybridization techniques. Variant nucleotide sequences also include synthetically derived nucleotide sequences that have been generated, for example, by using site-directed mutagenesis, but which still remain functionally equivalent.

With respect to the amino acid sequences for the various full length or mature polypeptides used in the vector system of the present invention, variants include those polypeptides that are derived from the native polypeptides by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Such variants may result from, for example, genetic polymorphism or from human manipulation. Methods for such manipulations are generally known in the art.

For example, amino acid sequence variants of a polypeptide can be prepared by mutations in the cloned DNA sequence encoding the specific vector element of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (2d ed., Cold Spring Harbor Laboratory Press, Plainview, N.Y.); U.S. Pat. No. 4,873,192; and the references cited therein; herein incorporated by reference. Guidance as to appropriate amino acid substitutions that may not affect biological activity of the various vector polypeptide may be found in the model of Dayhoff et al. (1978) Atlas of Polypeptide Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

A variant of a native nucleotide sequence or native polypeptide has substantial identity to the native sequence or native polypeptide. A variant may differ by as few as 1 to 10 amino acid residues, such as 6-10, as few as 5, as few as 4, 3, 2, or even 1 amino acid residue. A variant of a nucleotide sequence may differ by as low as 1 to 30 nucleotides, such as 6 to 20, as low as 5, as few as 4, 3, 2, or even 1 nucleotide residue.

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences can be accomplished using a mathematical algorithm. For example, percent identity of an amino acid sequence can be determined using the Smith-Waterman homology search algorithm using an affine 6 gap search with a gap open penalty of 12 and a gap extension penalty of 2, BLOSUM matrix 62. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic Version G. The Smith-Waterman homology search algorithm is taught in Smith and Waterman (1981) Adv. Appl. Math 2:482-489, herein incorporated by reference. Alternatively, the alignment program GCG Gap (Wisconsin Genetic Computing Group, Suite Version 10.1) using the default parameters may be used. The GCG Gap program applies the Needleman and Wunch algorithm and for the alignment of nucleotide sequences with an open gap penalty of 3 and an extend gap penalty of 1 may be used. Another preferred, non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences having sufficient sequence identity. BLAST protein searches can be performed with the XBLAST program, score=50, wordlength=3, to obtain amino acid sequences having sufficient sequence identity. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Within preferred embodiments of the invention, the DNA constructs described below are contained on a vector or plasmid. The plasmid may contain a bacterial origin of replication, one or more selectable markers, a signal that allows the plasmid construct to exist single stranded (i.e., a M13 origin of replication), a multiple cloning site, and a “mammalian” origin of replication (i.e., a SV40 or adenovirus origin of replication). Such vectors are known in the art.

A. Gene Transfer Vector

As noted above, the trans-viral vector system provides a gene transfer vector which in combination with the packaging construct, the env construct, and the trans-enzyme construct, which enable the construction of a trans-viral packaging cell line which precludes the formation of a replication competent virus.

As used herein the “gene transfer vector” refers to a nucleotide sequence that has the necessary “cis acting” components that allow for the transcription of the gene transfer vector; encapsidation of the gene transfer vector mRNA (i.e., modified proviral genome) into the viral particle; reverse transcription of the gene transfer vector mRNA; and integration of the gene transfer vector into the genome of the target cell.

Cis acting nucleic acids sequences that carry out the functions described above are well know in the art. For instance, a gene transfer vector can comprise the following components: a 5′ LTR; a packaging signal; a Rev Responsive Element (RRE); and a 3′ LTR or any functional variant or derivative of each of these elements. The gene transfer vector can further comprise at least one nucleotide sequence of interest operably linked to a promoter active in the desired target cell. Each of these components are described in more detail below.

The 5′ and 3′ LTR sequences flank the other elements of the gene transfer vector. The LTR sequences contain multiple elements including, for example, promoter/enhancer elements along with other cis-acting sequence elements important for integration and integration of the proviral genome into the genome of the target cell. Various cis-acting elements of the LTR include, for example, the U5 region (nt 97-181 of GenBank Accession No. AF033819) and the U3 region (nt 8631-9085 of GenBank Accession No. AF033819) which comprises viral promoter and enhancer sequences that direct the expression of the retroviral gene transfer vector into a single precursor mRNA. Other LTR components include the R region which comprises sequences required for RNA transcription initiation (i.e., the transactivating region (TAR)), the polyadenylation signals (nt 1-96 of GenBank Accession No. AF033819). A more complete description of LTR sequences and functional variants for the HIV-1 virus can be found in Pereira et al. (2000) Nucleic Acid Research 28:663-668, herein incorporated by reference. One of skill in the art will recognized that the various components of the gene transfer vector can be arranged in any order as long as they are internal to the 5′ and 3′ LTR elements. The transfer vector can further comprise tRNA primer binding site sequences (nt 182-199 of GenBank Accession No. AF033819 which functions in the initiation of reverse transcription. Such alterations are known to one of skill in the art. It is further recognized that modifications to the LTRs can be made, such as those of the self-inactivating (SIN) vectors. Such alterations are known to one of skill in the art.

As used herein the “packaging signal” or “ψ signal” refers to a nucleic acid sequence that is required in cis for the encapsidation of the viral RNA into the viral particle. The packaging signal used in the methods of the present invention may be a minimal packaging signal required for encapsidation of the gene transfer vector into the viral particle. This minimal packaging sequence for the preferred retroviral gene transfer vector of the present invention will be sufficient to direct the incorporation of the modified proviral genome (i.e., gene transfer vector) into the viral particle.

It is recognized that variants or fragments of known packaging signals may be used in the methods of the present invention so long as the variants direct the encapsidation of the retroviral gene transfer vector into the viral particle. For instance, extended packaging signals which encompass sequences surrounding the minimal packaging sequence may increase the efficiency of encapsidation of the gene transfer vector in the viral particle. The HIV packaging signal has been further characterized in Mcbirde et al. (1997) J. Virol. 71:4544-4554, which is herein incorporated by reference.

The gene transfer vector may also contain a Rev Responsive Element (RRE). The presence of this element allows Rev to direct the nuclear export of the RRE-containing mRNAs. The sequence of the RRE and functional variants thereof are known in the art. See, for example, Berchtold et al. (1995) Virology 211:285-289; Dillon et al. (1990) J. Virol. 64:4428-4437; Le et al. (1990) Nucleic Acid Research 18:1613-1623; all of which are herein incorporated by reference. It is further recognized that Rev/RRE can be substituted with other elements, including for example, the cis-acting 219-nucleotide constitutive transport element (CTE) from the Mason-Pfizer monkey virus (MPMV) that has been shown to allow Rev-independent HIV-1 replication. See, for example, Bray et al. (1994) Proc. Natl. Acad Sci USA 91:1256-1260.

The gene transfer vector further comprises a DNA construct having a nucleotide sequence of interest. Thus, the nucleotide sequence of interest contained in the gene transfer vector, when introduced into the target cell via the viral particle, can be expressed. In the methods and compositions of the present invention, the nucleotide sequence of interest has retinal therapeutic properties. The nucleotide sequence of interest may be native or heterologous (i.e., foreign) to the target cell. This sequence is contained in a DNA construct comprising all of the elements necessary for expression of a nucleotide sequence of interest in the target cell (i.e., a mammalian retinal cell). Thus, the nucleotide sequence of interest contained in the gene transfer vector, when introduced into the target cell via infection of the viral particle, can be expressed.

By “operably linked” is intended the individual nucleotide sequences are joined such that expression of the nucleotide sequence of interest is under the regulatory control of the 5′ and 3′ regulatory sequences. When the nucleotide sequence of interest encodes a polypeptide, “operably linked” further encompasses the joining of the nucleotide sequences such that expression of the coding sequences occurs in the proper reading frame. The gene transfer vector may contain at least one additional nucleotide sequence of interest operably linked to a promoter.

As used herein, the DNA construct containing the nucleotide sequence of interest will include in the 5′-3′ direction of transcription, a transcriptional and translational initiation region, a nucleotide sequence of interest, and a transcriptional and translational termination region functional in the targeted host cell (i.e., a mammalian retinal cell). The transcriptional initiation region, the promoter, may be native or foreign to the target cell. Additionally, the promoter may be the natural sequence or, alternatively, a synthetic sequence. By “foreign” is intended that the transcriptional initiation region is not found in the target cell into which the trans-viral vector is introduced. While it may be preferable to express the sequences using heterologous promoters, the native promoter sequence may be used. The termination region may be native with the transcriptional initiation region, may be native with the operably linked DNA sequence of interest, or may be derived from another source.

Any promoter may be operably linked to the nucleotide sequence of interest so long as the promoter is active in the target cell. Such promoters may be constitutive promoters (i.e., CMV promoter, SH VTK promoter, RSV promoter, beta actin promoter, and the SV40 promoter) or the promoter may be preferably expressed in the cells of the eye, more particularly the cells of retina. Such tissue-preferred promoters include PGK, those driving expression of Tissue Inhibitor of Metalloproteinases 3 (TIMP-3) tyrosinase, and RPE65. See, for example, Zeng et al.(1998) Developmental Dynamics 211:228-237; Ohta et al. (1996) Nuc. Acids Res. 24:938-942; and, Nicoletti et al. (1998) Invest. Ophthal. & Vis. Sci. 39:637-644. In one embodiment, the nucleotides sequence of interest is placed downstream of the CMV immediate early enhancer/chicken beta-actin promoter-exon1-intron element, which was followed by a poliovirus internal ribosomal entry sequence. See, for example, Acland et al. (2001) Nature Genetics 28:92-95.

It is further recognized that the DNA construct may contain various sequences that facilitate the expression, stabilization, and/or localization of the nucleotide sequence of interest and/or the resulting gene product. Such sequences include enhancers, introns, and post-transcriptional elements such as the Woodchuck Hepatitis Virus post-transcriptional region (WPRE) or PPT-CTS or functional variants thereof. See, for example, Zufferey et al. (1999) J. Virol. 73:2886-2892 and U.S. patent application Ser. No. 09/709,751, filed Nov. 10, 2000, both of which are herein incorporated by reference. In yet other embodiments the gene transfer vector further includes affinity tags for purification or labeling (e.g., with antibodies).

The gene transfer vector may further contain at least one DNA construct comprising a selectable marker operably linked to a promoter. Selectable markers include, but are not limited to, luciferase, β-gal, GFP, and various antibiotic resistance sequences. One of skill will appreciate that numerous possibilities exist. It is further recognized that the promoter selected for the expression of selectable marker will vary depending on if the marker is being used to monitor incorporation of the gene transfer vector into the packaging cell line or into the genome of the target cell.

In preparing the DNA construct, the various DNA fragments may be manipulated, so as to provide for the DNA sequences in the proper orientation and, as appropriate, in the proper reading frame. Toward this end, adapters or linkers may be employed to join the DNA fragments. For this purpose, in vitro mutagenesis, primer repair, restriction, annealing, resubstitutions, e.g., transitions and transversions, may be involved.

FIGS. 1-3 illustrate non-limiting examples of a gene transfer vector from the trans-viral system. The construct illustrated comprises the LTR sequence from HIV-1 flanking the following components: a packaging signal; an RRE; a central polypurine tract (PPT) and a nucleotide sequence of interest operably linked to the CMV promoter.

B. Trans-Enzyme Construct

The trans-enzyme construct contains the nucleotide sequences encoding Reverse Transcriptase and Integrase apart from their native configuration. In particular, the trans-enzyme construct encodes a fusion protein comprising a first polypeptide characterized by the ability to be targeted to a viral particle, operably linked to functional Reverse Transcriptase and/or Integrase polypeptide. For instance, an HIV virion-associated accessory protein (Vpr or Vpx) or a variant or fragment thereof can be used as a vehicle to deliver polypeptides having integrase activity and reverse transcriptase activity into the trans-viral vector particle. This “trans-lenti” viral vector design is illustrated in FIG. 1. Specifically, the trans-enzyme construct may comprise a nucleic acid sequence encoding a fusion protein comprising a Vpr or Vpx polypeptide or functional variant or fragment thereof, fused in frame to at least one heterologous polypeptide comprising Integrase and/or Reverse Transcriptase or functional variants and fragments thereof. Such trans-enzyme constructs are know in the art. See, for example, Liu et al. (1997) J. Virol. 71:7704-7710, Wu et al. (1997) EMBO J. 16:5113-5122; U.S. Pat. No. 6,001,985; U.S. patent application Ser. Nos. 09/089,900 filed Jun. 3, 1998; and, 09/460,548 filed Dec. 14, 1999; all of which are herein incorporated by reference.

As used herein, by “fusion protein” is intended a polypeptide having at least two heterologous polypeptide sequences joined for in-frame expression. That is, the nucleotide sequences encoding the heterologous polypeptides will be translated into a single translation product. In one embodiment, the fusion protein encoded by the trans-enzyme construct comprises a Vpr or a Vpx polypeptide or a functional fragment or variant thereof, while the second polypeptide comprises a polypeptide having integrase or reverse transcriptase activity. In other embodiments of the present invention, the fusion protein encoded by the trans-enzyme construct comprises nucleotide sequences encoding all three polypeptides (i.e., Vpr/Vpx, Reverse Transcriptase, and Integrase).

Sequences encoding Vpr and Vpx polypeptides are known in the art. See, for example, U.S. Pat. No. 5,861,161, herein incorporated by reference. Fragments and variants of a Vpr or Vpx polypeptide can be used and will retain the ability to be incorporated into virion particles. Examples of fragments and variants of the Vpr/Vpx polypeptides that retain this activity are known. See, for example, Paxton et al. (1993) J. Virol. 67:7229-7237 and U.S. Pat. No. 6,043,081, both of which are herein incorporated by reference. In addition, assays to determine if a Vpr or Vpx polypeptide are incorporated into a virion are routine in the art. Briefly, a fragment or variant of a Vpr/Vpx polypeptide fused to a marker polypeptide is expressed in a packaging cell line capable of producing viral particles. The cell line is cultured and viral particles are produced. The viral particles are isolated and assayed for the presence of the marker protein. See, for example, Paxton et al. (1993) J. Virol. 67:7229-7237.

Another embodiment of the trans-vector design is illustrated in FIG. 2. The design in this particular non-limited example is referred to herein as the trans-retoviral vector design that uses a fragment of the Gag polypeptide which retains the ability to be targeted to the viral particle to deliver the polypeptides having integrase activity and reverse transcriptase activity into the trans-viral vector particle.

Specifically, in this embodiment, the trans-enzyme construct may comprise a nucleic acid sequence encoding a fusion protein comprising a fragment of a Gag polypeptide which retains the ability to be targeted to the viral particle, fused in frame to at least one heterologous polypeptide comprising Integrase and/or Reverse Transcriptase or functional variants and fragments thereof. Such trans-enzyme constructs are known in the art. See, for example, U.S. patent application Ser. No. 09/578,548, filed Dec. 14, 1999.

In one embodiment, the fusion protein encoded by the trans-enzyme construct comprises a fragment of a Gag that retains the ability to be targeted to the viral particle, while the second polypeptide comprises a polypeptide having integrase or reverse transcriptase activity. In other embodiments of the present invention, the fusion protein encoded by the trans-enzyme construct comprises nucleotide sequences encoding all three polypeptides (i.e., Gag:Reverse Transcriptase: Integrase).

Sequences encoding Gag polypeptides are known in the art. Fragments and variants of the Gag polypeptide will retain the ability to be incorporated into virion particles. Examples of fragments and variants of the Gag polypeptides that retain this activity are known.

Nucleotide sequences encoding Integrase polypeptides are also known (see Table 1). Integrase is involved in many aspects of the viral life cycle. For instance, Integrase has been shown to be involved in various steps of the virion assembly and maturation process and is involved in reverse transcription and integration of the viral genome in the host cell (Lie et al. (1999) J. Virol. 73:8831-8836 and Craigie et al. (2001) J. Bio. Chem. Manuscript RI 00027200). The Integrase polypeptide used in the methods of the present invention may be from any viral source, particularly a retrovirus, particularly a lentiviral source.

The Integrase polypeptide or variants or fragments thereof will have integrase activity. By integrase activity is intended the polypeptide retains sufficient activity to support the production of a viral particle (i.e., supports virus assembly, maturation, and/or integration). The regions of the Integrase polypeptide that influence virus assembly, maturation, and integration are known in the art, as are assays to determine these functions. For instance, mutations in the catalytic center of Integrase decreases infectivity of the viral particle. See, for example, Liu et al. (1997) J. Virol. 71:7704-7710; Wu et al. (2000) Mol. Therapy 2:47-55; and Craigie et al. (2001) J. Bio. Chem. Manuscript RI 00027200 and Liu et al.(1999) J. Virol. 23:8831-8836; all of which are herein incorporated by reference.

Nucleotide sequences encoding Reverse Transcriptase are known in the art (see Table 1). Reverse Transcriptase is involved in the synthesis of double stranded, linear DNA from a single-stranded RNA template using cellular tRNA as a primer. The Reverse Transcriptase used in the present invention may be from any retroviral source, particularly a lentivirus including, but not limited to, HIV-1, HIV-2, and SIV. The Reverse Transcriptase polypeptide or variant or fragment thereof will have reverse transcriptase activity.

By “reverse transcriptase activity” is intended the polypeptide retains sufficient activity to support the production of a viral particle (i.e., catalyze replication of the gene transfer vector). Assays to measure reverse transcriptase activity are known in the art. For example, measurements of reverse transcriptase activity can be carried out in-vitro using an artificial template/primer construct and tritiated deoxynucleotide triphosphate as the nucleotide substrate. This assay system is based on detecting incorporation of radioactivity in RNA/DNA hybrids that can be precipitated with trichloroacetic acid (TCA). Other methods for determining reverse transcriptase activity can be found in, for example, U.S. Pat. No. 6,132,995 herein incorporated by reference.

It is well within skill in the art to generate an expression vector having at least two nucleotide sequences encoding heterologous polypeptides that will be translated into a single translation product (i.e., fused in frame). Furthermore, one of skill in the art will recognize that the linkers may be placed between the nucleotides sequences that encode the heterologous polypeptides of the trans-enzyme construct, so long as the linker sequence allows the coding regions of the peptides to remain in frame. Such linker sequences include, for example, protease cleavage sites that are recognized by the protease encoded on the packaging construct. Such sequences are known in the art and include, for example, the 33 nucleotides 5′ to the Reverse Transcriptase coding sequence of the HIV-1 genome or alternatively, the 18 nucleotides located 5′ to the Integrase coding sequence of GenBank Accession No. L02317. See, for example, Wu et al. (1997) EMBO J. 16:5113-5122. One of skill in the art will recognize how to use such sequences to result in effective cleavage of the Reverse Transcriptase or Integrase from the Vpr or Vpx polypeptide.

To produce a replication defective trans-viral particle both Integrase and Reverse Transcriptase are expressed in the packaging cell line in trans to the packaging construct which encodes the Gag or Gag/Pro polypeptide. In one embodiment of the present invention, fusion proteins comprising the Reverse Transcriptase and Integrase are expressed in the packaging cell lines on two separate trans-enzyme constructs. In this embodiment, the packaging cell line will contain at least two trans-enzyme constructs. Using the trans-lentiviral vector design as an example, the first fusion protein comprising Vpx or Vpr and Reverse Transcriptase, and the second trans-enzyme construct encoding a fusion protein comprising Vpx or Vpr and Integrase. It is recognized that the order of the polypeptides in these constructs may vary. Moreover, it further recognized that the trans-retroviral vector design can be used in a similar manner.

In another embodiment of the trans-enzyme construct, the polypeptides encoding the Reverse Transcriptase and Integrase or variants or fragments thereof are expressed as a single translation product. In this embodiment, the trans-enzyme construct encodes a fusion protein having the following polypeptides or functional variants or fragments thereof fused in frame: Vpr or Vpx, Reverse Transcriptase, and Integrase. See, for example, U.S. patent application Ser. No. 09/089,900 and Wu et al. (1997) EMBO J. 16:5113-5122.

The nucleotide sequence encoding the fusion protein of the trans-enzyme construct is operably linked to a promoter active in the packaging cell line. The DNA construct containing the trans-enzyme construct may further comprise transcriptional and translational termination regions that are also functional in the packaging cell line. Promoters of interest include, for example, the chicken beta actin promoter CMV, HIV-2 LTR, the SHVTK promoter, the RSV promoter, the adenovirus major late promoter and the SV 40 promoters.

FIG. 1 illustrates one non-limiting example of a trans-enzyme construct of the trans-lentiviral vector system. The construct comprises the following operably linked components: a CMV promoter; a nucleotide sequence encoding a Vpr polypeptide; a nucleotide sequence encoding Reverse Transcriptase; a nucleotide sequence encoding Integrase; the RRE from HIV-2; and a SV40 polyadenylation signal. The trans-enzyme construct illustrated in FIG. 1, preserves the N-terminal protease cleavage site of Reverse Transcriptase and the protease cleavage site between the Reverse Transcriptase and Integrase polypeptides.

C. Packaging Construct

As noted above, the trans-viral particle used in the methods of the present invention further provides a packaging construct, which in combination with the gene transfer vector, the env construct, and the trans-enzyme construct, enable the construction of a packaging cell line which precludes the formation of a replication competent virus.

The packaging construct is characterized as a nucleic acid sequence comprising at least one nucleotide sequence that encodes a truncated Gag/Pol sequence (i.e., Gag/Pro) that does not encode a functional Integrase or Reverse Transcriptase polypeptide. A nucleotide sequence that encodes a Gag/Pro polypeptide comprises a variety of structural proteins that make up the core matrix and nucleocapsid polypeptides. The sequence further encodes a functional protease. The Gag/Pro sequences may be derived from any retrovirus as described elsewhere, herein. Such Gag/Pro sequences are known in the art and include, for example, nucleotides 336-2099 of Genbank Accession No. AF033819.

The packaging construct can further contain nucleotide sequences from a viral genome particularly the retroviral genome) which are necessary for the production of a replication defective viral particle. Examples of genetic elements that may be contained in the packaging construct include, but are not limited to, nucleotide sequences encoding Vif, Tat, and Rev. The packaging construct, however, does not contain nucleotide sequences which encode a functional Envelope polypeptide, a functional Reverse Transcriptase polypeptide, and a functional Integrase polypeptide. These sequences have been totally or partially deleted or alternatively, have been altered to prevent translation of a functional polypeptide. Furthermore, the packaging construct lacks a functional packaging signal (ψ signal) thereby preventing the RNA produced from this construct from being incorporated into the viral particle.

As explained in more detail in U.S. patent application Ser. No. 09/089,900, the manner in which the Reverse Transcription and Integrase are mutated in the packaging construct may affect the infectivity of the viral particle. It is recognized that any alteration can be made to the reverse transcriptase and/or integrase sequence in the packaging construct that disrupts the function of the polypeptides and still allows for the production of an infectious, replication defective trans-viral vector when the Reverse Transcriptase and Integrase are expressed in trans in the trans-enzyme construct. For instance, in one embodiment, the reverse transcriptase and integrase sequences are altered to contain a stop codon 3′ to the protease sequence. It is further recognized that multiple mutations maybe introduced into the reverse transcriptase and integrase sequence. For example, in addition to introducing a translation stop early in the coding sequence of Reverse Transcriptase, at least one additional “fatal” mutation can be positioned within the Reverse Transcriptase and/or Integrase coding sequence. This additional mutation further decreases the likelihood of reestablishing a complete Gag-Pol coding region by genetic recombination between the packaging construct and the trans-enzyme construct.

The nucleotide sequences of the packaging construct are contained in a DNA construct which further comprises a promoter active in the packaging cell line. The DNA construct may further comprise transcriptional and translational termination regions which are also functional in the packaging cell line. Promoters of interest include, for example, CMV, HIV-2 LTR, the HCMV-IE (Naldini et al. (1996) Science 272:263-267), the SHVTK promoter, the RSV promoter, the adenovirus major late promoter and, the SV40 promoters. The packaging construct may further contain a selectable marker operably linked to an active promoter.

One of skill in the art will recognize that various functional variants of the packaging construct can be envisioned. FIGS. 1-3 illustrate a non-limiting example of a packaging construct useful in the methods of the present invention. The construct comprises a CMV promoter operably linked to a nucleotide sequence encoding Gag/Pro, Vif, Tat, Rev (nt 258-8384 of Genbank Accession No. L02317). The packaging construct of further comprises translational stop codons (TAA) at the first amino acid position of the Reverse Transcriptase and Integrase coding sequences, a deletion of the ψ signal, a frame shift mutation in the Vpr coding sequence, a complete deletion of the nef gene, an internal deletion that results in an inactive Vpu polypeptide, and a deletion of the nucleotides which encode the Env polypeptide. For a more detailed description of the construction of the packaging construct see U.S. patent application Ser. No. 09/089,900.

D. Env Construct

The present invention further provides an env construct that in combination with the packaging construct, gene transfer vector, and trans-enzyme construct described above, preclude formation of a replication complete trans-virus vector particle.

The env construct of the trans-viral system comprises a nucleotide sequence encoding an envelope protein or a functional variant or fragment thereof operably linked to an active promoter. A variety of envelope polypeptides are known in the art. It is recognized that the host range of cells that the viral particles of the present invention can infect can be altered depending on the envelope coding sequence used. Viral envelope proteins useful in the present invention include HIV envelope polypeptides (see Table 1), the MLV envelope glycoprotein (Page et al. (1990) J. Virol. 64:5270-5276), the vesicular stomatitis virus G-protein (VSV-G) (Yee et al. (1994) Proc. Natl. Acad. Sci. 91:9564-9568 and Burns et al. (1993) Proc. Natl. Acad. Sci. USA 90:8033-8037) or the envelope polypeptides of Ebola and Makola (Kobinger et al. (2001) Nature Biotechnology 19: 225-230). In preferred embodiments of the present invention, the env coding sequence chosen will allow for the entry of the viral particle into the cells of the mammalian eye, particularly a retinal cell, more particularly an RPE. In the methods of the present invention, the G-protein of vesicular-stomatitis virus (VSV-G) or a fragment or variant thereof is used in the env construct. Pseudotyping assays to determine additional envelope polypeptides useful in the methods of the present invention are well known in the art. It is further recognized that the trans-viral vector particle may be constructed in the absence of the Env construct.

A fragment or variant of an Envelope polypeptide will retain sufficient activity to support the production of a replication defective viral particle (i.e., capable of being incorporated into the envelope of a retroviral particle and capable of binding to target cells and allowing entry of the viral particle into the target cells). Assays for determining the function of an Env polypeptide or a fragment or variant thereof are known in the art. For example, expression of a fragment or variant of an Env polypeptide of the present invention will allow vector particles produced in that packaging cell line, to transmit a selectable marker to a naive sensitive cell such that it becomes resistant to the marker drug selection.

Any promoter sequence may be used in the env construct, so long as it is active in the packaging cell line. Such promoter sequences have been described elsewhere herein. Representative examples of suitable polyadenylation signals include the SV40 late polyadenylation signal, the bovine growth hormone termination/polyadenylation sequence, and the insulin polyadenylation signal.

The env construct may further comprise a nucleotide sequence encoding a selectable marker. Examples of such selectable markers include nucleotide sequences capable of conferring host resistance to antibiotics (e.g., puromycin, ampicillin, tetracycline, kanamycin, and the like), or conferring resistance to amino acid analogues, etc. Other selectable markers are well known in the art, including for example βgal, GFP, and luciferase. One of skill will appreciate the numerous possibilities.

As an illustrative and non-limiting example of the env construct of the present invention, is shown in FIG. 1. The construct comprises the CMV immediate early promoter operably linked to a VSV-G coding region operably linked to an SV40 polyadenylation signal.

III. Packaging Cell Lines

The trans-viral particles used in the methods of the present invention are generated using techniques known in the art. See, for example, U.S. Pat. No. 4,650,764, and U.S. patent application Ser. No. 09/089,900 here incorporated by reference. The methods include incorporating into a packaging cell line the trans-enzyme construct, the gene transfer construct, the env construct, and the packaging construct; culturing the packaging cell line under suitable conditions that allow for the formation of viral particles; and, isolating the trans-viral particles. A wide variety of animal cells may be used to prepare the packaging cells of the present invention, including, for example, cells obtained from vertebrates, or mammals such as human, feline, goat, bovine, sheep, dog, and mice. Suitable packaging cell lines include, but are not limited to, HeLa (ATCC No. CCL2); HT1080 (ATCC No. CCL121); 293 (ATCC No. 1573); and the 293T cell line.

The various vector constructs may be introduced into the packaging cells through any means known in the art. Such means include, but are not limited to, electroporation, the use of liposomes, and CaPO₄ precipitation. See, for example, Ausabel et al. (1994) Current Protocols in Molecular Biology; John Wileg and Sons, Inc., and U.S. Pat. No. 5,739,081, both of which are herein incorporated by reference. It is further recognized that the various constructs of the trans-viral system can be transiently expressed in the packaging cell line, or alternatively, any or all of the constructs can be stably incorporated into the genome of the packaging cell.

Methods for culturing the packaging cell line under conditions in which the trans-viral particle is produced and the subsequent isolation of retroviral vector particles are also known in the art. For instance, the retroviral vector packaging cell lines may be cultured using standard culturing techniques, including any of a variety of monolayer culture systems. Such packaging cell lines may be cultured in T-flasks, roller bottles, or bioreactors. Any acceptable culture medium may be used, such as, AIM-V medium (Gibco BRL, Grand Island, N.Y) containing 5% fetal bovine serum, or Dulbecco's modified Eagle medium (DMEM) with high glucose (4.5 g/l) supplemented with 10% heat-inactivated fetal bovine serum

Methods of isolating retroviral vector particles are known in the art, see for example, U.S. Pat. Nos. 5,661,022 and 6,013,517; herein incorporated by reference. As used herein, “purified trans-virus vector particles” means a preparation of trans-viral vector particles containing at least 50%, 60%, 70%, 80% by weight, preferably at least 85% by weight, and more preferably at least 90%, 93%, 95%, 98%, 99% by weight, of the retroviral vector particles.

IV. Pharmaceutical Formulations and Methods of Administration

As discussed above, the trans-viral vector of the present invention is used for the delivery of a nucleotide sequence of interest to a retinal cell of a mammal. In this manner, the level of a nucleotide sequence of interest in a retinal cell of the mammal is modulated (i.e. increased or decreased). Methods of administering a trans-viral particle to a mammalian retinal cell in order to allow for the modulation in the level of the nucleotide sequence of interest are well known in the art. Any means of administering (i.e., contacting) the retinal cell with the viral particle that permits the viral particle to infect the target cell may be used. In this embodiment, the effective dose administered to the retinal cells will be sufficient to allow for the integration of the nucleotide sequence of interest and to produce the desired modulation of the level/activity of the nucleotide sequence and/or the protein it encodes.

In other embodiment of the present invention, the delivery and expression of the nucleotide sequence of interest in a mammalian retinal cell finds use in the improvement of the clinical outcome of a mammal having a retinal disorder. In this embodiment, delivery of a therapeutically effective amount of a trans-viral vector particle of the present invention to a target cell of a mammal (i.e., a retinal cell, more particularly an RPE cell) may be accomplished via administration of a pharmaceutical composition comprising a therapeutically effective dose of this viral vector particle. By “therapeutically effective amount” or “dose” is meant the concentration of viral vector particle that is sufficient to elicit the desired therapeutic effect (i.e., an improvement in either the rate or the extent of behavioral recovery of the mammal having a retinal degenerative disorder or an improvement in the morphological and/or electrophysiological preservation of the mammals photoreceptors). As such, a therapeutically effective dose will be sufficient to reduce or lessen the clinical symptoms of the retinal degenerative disorder being treated or prevented.

Methods to quantify if the retinal degenerative disorder has been treated or prevented are well known to those skilled in the art. Such methods include, but are not limited to, histological methods, electrophysiological assays, and functional/behavior analysis. For example, morphologic and electrophysiological preservation can be monitored as a delay in the number of photoreceptor nuclei lost, assessment of photoreceptor layer thickness (i.e., counting the number of rows of photoreceptor nuclei and the widths of the outer and inner segments in injected portions of the retina and comparing the counts to a non-injected retina), and the enhancement of rod electroretinographic (ERG) amplitudes. See, for example, Acland et al. (2001) Nature Genetics 28:92-95. In addition, qualitative visual assessment techniques may also be used to assay an improvement in the condition of the mammal.

The therapeutically effective amount will depend on many factors including, for example, the retinal degenerative disorder being treated and the responsiveness of the subject undergoing treatment. Methods to determine efficacy and dosage are known to those skilled in the art.

A therapeutically effective amount of a viral particle of the present invention comprises from about 5×10⁷ to about 4×10³ virus particles, from about 4×10⁶ to about 5×10⁴ infectious virus particles, from about 4×10⁵ to about 5×10⁵ infectious virus particles, or from about 4×10⁷ to about 5×10⁷ infectious virus particles.

It is further recognized that in specific embodiments of the present invention, lower viral titers can be used. A “sufficient concentration” of virus will allow for the integration of the nucleotide sequence of into a single target cell. Methods to assay for such an event are well known in the art.

It is further recognized that the therapeutically effective dose of the trans-viral vector particle may be administered intermittently. By “intermittent administration” is intended administration of a therapeutically effective dose of a trans-viral vector of the invention, followed by a time period of discontinuance, which is then followed by another administration of a therapeutically effective dose, and so forth. The preferred length of the discontinuance period depends on the disorder being treated. The discontinuance period can be at least 1, 2, 4, 6, 8, 10, 12, 14, 16, 18, 20, 22, 24 months or greater. An intermittent schedule of administration of the trans-viral vector particle can continue until the desired therapeutic effect is achieved. Subjects which may be administered the trans-virus of the invention include, but are not limited to, humans and animal (e.g., pig, cattle, dog, horse, donkey, mouse, hamster and monkey) subjects.

By “pharmaceutically acceptable carrier” is intended a carrier that is conventionally used in the art to facilitate the storage, administration, and/or the biological activity of a viral vector particle. A suitable carrier should be stable, i.e., incapable of reacting with other ingredients in the formulation. It should not produce significant local or systemic adverse effect in recipients at the dosages and concentrations employed for treatment. Such carriers are generally known in the art. Exemplary pharmaceutically acceptable carries include, but are not limited to, sterile pyrogen-free water and sterile pyrogen-free physiological saline solution. The formulations may be conveniently prepared in unit dosage forms and may be prepared by any of the methods known in the art.

In one embodiment of the present invention, the viral particle is administered to a mammal to treat or prevent a retinal degenerative disorder. In specific embodiments, the trans-viral vector is administered directly to the retina via techniques know in the art. For example, subretinal injection, using a transscleral transchoroidal approach may be used. See, for example, Bennett et al. (1997) Invest. Ophthalmol. Vis. Sci. 35:2535 and Bennett et al. (1997) Invest. Opthalmol. Vis. Sci. 38:2857, both of which are herein incorporated by reference. Subretinal injections can also be accomplished in the larger eye of rabbits, non-human primates, and ultimately humans under direct visualization using an anterior approach.

In another embodiment of the invention, methods are provided for the ex vivo transfer of the nucleotide sequence of interest having retinal therapeutic properties to the mammalian retinal cells. Such methods comprise removing mammalian retinal cells and culturing them in-vitro; infecting the isolated retinal cells with the trans-viral vector particle; and, delivering the infected cells to the retina of a mammal. It is further recognized that the isolated retinal cells may be returned to the same animal or may be delivered to another allogenic animal. In such an instance, it is preferable to have histocompatibility-matched animals.

In one embodiment of the present invention, the methods and compositions find use in the treatment/prevention of LCA. LCA causes near total blindness in infancy and can result from mutations in RPE65 (LCA, type II; MIN 180069 and 204100). As used herein, a “therapeutically effective amount” of a trans-viral particle for the treatment of LCA will be sufficient to reduce or lessen the clinical symptoms of the retinal degenerative disorder. As such, a therapeutically effective amount of a trans-viral particle will improve the clinical status of the treated animals in comparison to control animals that did not receive the therapy. Improvement in the clinical status for LCA includes, for example, improved retinal function (i.e., assayed by ERG photoresponses), improved transmission of retinal activity to higher visual pathways (i.e., assayed by pupillometry); and/or qualitative visual assessments (i.e., behavioral testing). Such assays are known in the art and are described in more detail in Acland et al. (2001) Nature Genetics 28:92-95. Such assays can be readily used by one skilled in the art to determine the dosage range of the viral vector for the effective treatment of LCA.

### EXPERIMENTAL

### EXAMPLE 1

### Favorable Immune Response Following Subretinal Administration of Lentivirus

I. Materials and Methods:

A. Preparation of the Trans-Lentiviral Vector

Plasmids: To construct the pPCW-eGFP gene transfer vector, a PCR amplified DNA fragment containing the EGFP cDNA (derived from pEGFP-C1, Clontech) was ligated into the BamHI/XhoI sites of the pHR-CMV-LacZ plasmid (Naldini et al. (1996) Science 272:263-8), generating pHR-CMV-eGFP. Then, a 150 bp sequence of DNA (coordinates 4327 to 4483) containing the central polypurine tract (PPT) and central terminal site (CTS) was PCR-amplified from the HIV-1 pSG3 molecular clone (Ghosh et al. (1993) Virology 194:858-864) and ligated into the unique ClaI site of pHR-CMV-eGFP. To increase eGFP expression, a post transcriptional regulator element derived from the woodchuck hepatitis virus (WPRE) (Zufferey et al. (1999) J. Virol. 7:2886). was inserted down-stream of eGFP, generating the pPCW-eGFP gene transfer vector.

B. Preparation of Trans-Lentiviral Vector Stocks

Stocks of the trans-lentivirus were produced by transfecting 5 μg of the pCMV-gag-pro packaging plasmid, 1.5 μg of the pCMV-vpr-RT-IN trans-enzyme plasmid, 2 μg of the pMD.G (VSVG) expression plasmid (env construct), and 5 μg of the gene transfer (vector) plasmid into subconfluent monolayer cultures of 293T cells by the calcium phosphate DNA precipitation method. Supernatants were harvested after 60 h, clarified by low-speed centrifugation (1000 g, 10 min), filtered through 0.45-μm pore-size filters, aliquoted, and frozen at −80° C. To determine vector titers, supernatant stocks of stocks of 0.2, 0.04, 0.008, 0.0016, 0.00032, and 0.000064 μl were used to infect cultures of HeLa cells, and GFP-positive (green) cell colonies were counted 2 days later using a fluorescence microscope. Each GFP-positive cell colony was measured as a single infectious unit (IU). The titer of the purified virus used was 4×10⁸ IU/ml. Aliquots of virus were stored at −80° C. until use.

C. Animals

Immunocompetent C57B1/6 mice aged 6 to 8 weeks (Jackson Labs, Bar Harbor, Me.) were housed under 12 hour light/12 hour dark conditions. The mice were maintained as outlined in the Association for Research in Vision and Ophthalmology (ARVO) Statement for the Use of Animals in Ophthalmic and Vision Research, and according to federal, state, and local regulations. The minimal number of animals was used to obtain statistically significant results.

D. Intraocular Administration of Trans-Lentivirus

Virus was administered bilaterally through subretinal injection, using a transscleral transchoroidal approach as previously described (Bennett et al. (994) Invest. Ophthalmol. Vis. Sci 35:2535; Bennett et al. Invest. Ophthalmol. Vis. Sci. 38:2857). One microliter of virus-containing solution (containing a total of 4×10⁵ IU) was injected, creating a dome-shaped retinal detachment, which was visible under the microscope.

E. In vivo Assessment of GFP

Ophthalmoscopy was used to monitor the presence and extent of GFP expression as described (Bennett et al. (1997) Invest. Ophthalmol. Vis. Sci. 38:2857; Dudas et al. (1999) Vis. Res. 39:2545). Evaluations were initiated on the day following subretinal injection and were continued daily for 9 days and then at weekly intervals. Fundus photographs were obtained using a Kowa Genesis camera (Keeler Instruments, Broomall, Pa.) equipped with a Wratten 47B gelatin excitation filter (Edmund Scientific, Barrington, N.J.).

The extent of retinal fluorescence was assessed with a numerical grading system ranging from 0 to 4, as described previously (Bennett et al. (1997) Invest. Ophthalmol. Vis. Sci. 38:2857). Briefly, grade 0 represents no fluorescing cells; grade 1, isolated fluorescing cells, giving a speckled appearance; grade 2, non-confluent patches of fluorescing cells 1-2 disc areas in size; grade 3, confluent patches of fluorescing cells several disc areas in size, but requiring a condensing lens to appreciate the fluorescence through the pupil; grade 4, same as grade 3, but fluorescence is visible through the pupil without the aid of a condensing lens. This grading system minimizes the inherent variability between injections and allows for direct comparisons between mouse eyes regardless of the exact portion of the retina that is transduced.

F. Histological Assessment of GFP Expression

Animals were sacrificed at time points ranging from 24 hours to 111 days after subretinal injection, and eyes were fixed in 4% paraformaldehyde in phosphate-buffered saline (PBS) for 24 hours. After cryoprotection in 30% sucrose in PBS, eyes were immersed in optimal cutting temperature compound (O.C.T.; Baxter, McGraw Park, Ill.) and frozen in a dry ice bath containing dimethylpentane. Serial sections were cut at 10 μm at a temperature of 23° C. using a Reichert-Jung cryostat (Bensheim, Germany). Sections were evaluated using a Leica DMXRE microscope, equipped with fluorescein isothiocyanate (FITC), rhodamine, and amber filters. The amber filter was used to distinguish GFP-induced fluorescence from background fluorescence as described (Anand et al. (2000) Hum. Gene. Ther. 11:449).

G. Assays of Cell Mediated Immune Responses

1. Histological Studies

Tissue sections were stained with hematoxylin and eosin and observed under bright field in order to assess the presence of inflammatory cells. Additional adjacent sections were subjected to immunohistochemical analyses in order to evaluate the presence of CD4-, CD8-, and CD16-positive cells. These sections were incubated with monoclonal rat anti-mouse antibodies (Boehringer Mannheim, Indianapolis, Ind.) specific for L3T4 (CD4) at a dilution of 1:50 or for Ly-2 (CD8a) at a dilution of 1:100 for 30 min at room temperature. The sections were washed with PBS and incubated for 30 min at room temperature with biotin-SP-conjugated goat anti-rat IgG (Jackson Immunoresearch Laboratories, West Grove, Pa.). Muscle tissue sections from animals that had received intramuscular injection of trans-lenti virus-GFP were used as controls. The sections were then incubated with Streptavidin-phycoerythrin (SIGMA Chem Co) (1:1000) for 45 minutes and mounted with vectashield (Vector Labs, CA).

2. Assay for Delayed-Type Hypersensitivity (In vivo Analysis)

DTH was used as an indicator of host cell-mediated responses to viral or transgene (GFP) antigens in vivo. Briefly, 7 days after subretinal injection of trans-lenti virus-GFP, right and left hind footpad thickness' of each animal were measured using an engineer's micrometer (Warren-Knight Instrument Co., Philadelphia, Pa.) and recorded. Antigen challenge was performed by injection of the right footpad intradermally, with either trans-lenti virus-GFP in PBS (50 μl of virus solution, containing 2×10⁵ IU of intact virus particles) or GFP solution alone in PBS (0.5 μg GFP in 50 μl of solution), which raised a visible intradermal wheal. The left footpad served as untreated control. Right footpad thickness' were measured 24 hours later for swelling. Control animals were sensitized by intramuscular injection of trans-lenti virus-GFP in the right gastrocnemius and tibialis anterior muscles. Leg hair was removed using Nair lotion hair remover with baby oil (Carter-Wallace, New York, N.Y.), and 50 μl of trans-lenti virus-GFP (stock diluted 1:10 in PBS, containing 2×10⁵ IU of virus) were injected. Footpad challenge with trans-lenti virus-GFP or GFP alone and thickness measurements were performed as described above. All measurements were performed in triplicate.

3. T-Cell Proliferation Assay (In vitro Analysis)

The induction of antigen-specific cytokines was analyzed at 24 hours and 7 days following subretinal trans-lenti virus-GFP injection using a T-cell proliferation assay (Elispot). Immunospot plates (Cellular Technology Ltd., Cleveland, Ohio) were coated with primary antibodies directed against the cytokines IL-2, IFN-γ, TNF-α, IL-4, IL-10, and TGF-β (PharMingen, San Diego, Calif.; 100 μl/well), and incubated overnight at 4° C. Primary antibodies were removed and the Elispot plates were blocked for 1 hour with 3% bovine serum albumin (BSA) in PBS. Animals were sacrificed by hypothermia and spleen-derived T cells were isolated and plated on the primary antibody-coated Elispot plates at a concentration of 1×10⁵ or 5×10⁵ cells/well (100 μl total volume/well, diluted in Dulbecco's modified Eagle's medium [DMEM]+10% fetal bovine serum [FBS]). The plates were then incubated for 48 hours at 37° C. under one of the following conditions: 1) in cell media alone (unstimulated control group); 2) in the presence of the T-cell stimulants Phorbol 12-Myristate 13-Acetate (PMA) and Ionomycin (positive control group; each stimulant diluted 1:100 in DMEM; final concentration 10 ng; Sigma, St. Louis, Mo.); 3) in the presence of trans-lenti virus antigens (trans-lenti virus-stimulated group; 2×10⁵ IU of trans-lenti virus-GFP diluted in DMEM); and 4) in the presence of GFP antigens (GFP-stimulated group; 0.1 μg GFP/well, diluted in DMEM). After 48 hours, cells were removed and the plates were washed three times with filtered PBS containing 0.1% Tween and then twice with filtered PBS alone. Secondary (biotinylated) antibodies against the cytokines listed above were added to the appropriate wells and the plates were incubated overnight at 4° C. Primary and secondary antibodies for all cytokines were diluted 1:500 in 3% BSA. The following day, secondary antibodies were removed and the Elispot plates were washed again in the manner described above, in order to remove non-specific binding of secondary antibodies. The tertiary antibody extravidin alkaline phosphatase (Sigma, St. Louis, Mo.; diluted 1:1000 in 3% BSA; 100 μl/well) was added to each well and the Elispot plates were again incubated overnight at 4° C. Following removal of tertiary antibody by the washing procedure described above, BCIP/NBT phosphatase substrate (Kirkegaard & Perry Laboratories, Gaithersburg, Md.), at room temperature, was added (100 μl/well) and the plates were left in the dark for 5-7 minutes. During this time they were monitored by a magnifying glass for the appearance of color-reactive spots. Plates were washed once with distilled water and left to dry overnight. The plates were read by Cellular Technologies, Ltd. (Cleveland, Ohio). The analysis of readings was done using Immunospot software provided by Cellular Technologies.

H. Assays of Humoral Immune Responses

1. Enzyme-Linked Immunosorbent Assay (ELISA)

Serum samples from mice injected either subretinally or intramuscularly with trans-lenti virus-GFP were analyzed for virus- and transgene-specific antibodies by enzyme-linked immunosorbent assay (ELISA) using a Wallac Victor2 1420 Multilabel Counter (Perkin Elmer, Gaithersburg, Md.). Baseline (Day 0) blood samples were obtained by tail vein puncture and then at 7 and 21 days following administration of trans-lenti virus-GFP, and serum was isolated. Samples were stored at −80° C. until the time of analysis. Enhanced protein-binding ELISA plates were coated overnight at 4° C. with antigen (purified trans-lenti virus-GFP, both intact and lysed, at 1:1000 dilution in bicarbonate buffer, pH 9.6). Alternatively, ELISA plates were coated with GFP (0.5 μg/50/μl buffer; Clontech) and then washed, blocked, and incubated with serum (diluted 1:100 in 3% BSA) overnight. Saline was used as negative control. Samples were then incubated with biotinylated anti-mouse antibodies to isotypes IgG₁, IgG₂ₐ, IgG_{2b}, and IgG₃ (100 μl/well; Pharmingen [San Diego, Calif.] and Becton Dickinson [Franklin Lakes, N.J.]) overnight. The wells were washed and incubated overnight with alkaline phosphatase-conjugated extravidin (100 μl/well; Sigma, St. Louis, Mo.), diluted 1:1000 in 3% BSA. The wells were washed again and the Sigma Fast Paranitrophenyl Phosphate Substrate system was used for the color reaction. Each reaction was performed in triplicate and the plates were read at an optical density of 405 nm. 2. Identification of Neutralizing Antibodies

In order to analyze neutralizing antibodies induced by subretinal and intramuscular injection of trans-lenti virus-GFP, 10 μl serum samples from each group of mice were plated in 96-well round-bottom tissue culture plates. Serial dilutions of each sample were made using serum-free DMEM. A constant amount of trans-lenti virus-GFP in serum-free DMEM (2×10⁵ IU) was added to each dilution to a total volume of 50 μl, and the plates were incubated for 1 hr at 37° C. The trans-lenti virus-GFP/serum mixtures were used to infect 84-31 cells (293 cells that express the E3 and E4 genes), which were already seeded at 60% confluence in 96-well flat-bottom tissue culture plates. The following day, the plates were inspected visually with a fluoroimager (Molecular Dynamics, Sunnyvale, Calif.). The intensity of GFP as assessed by fluorescence microscopy was directly proportional to the intensity of GFP detected by the fluoroimager. The latter is inversely proportional to the concentration of neutralizing antibodies. The neutralizing antibody titer was defined as the highest dilution that permitted GFP production to reach 50% of that in control samples (virus not pre-incubated with serum prior to infection).

II. Results

A. Assessment of Retinal Transduction and Transgene Expression

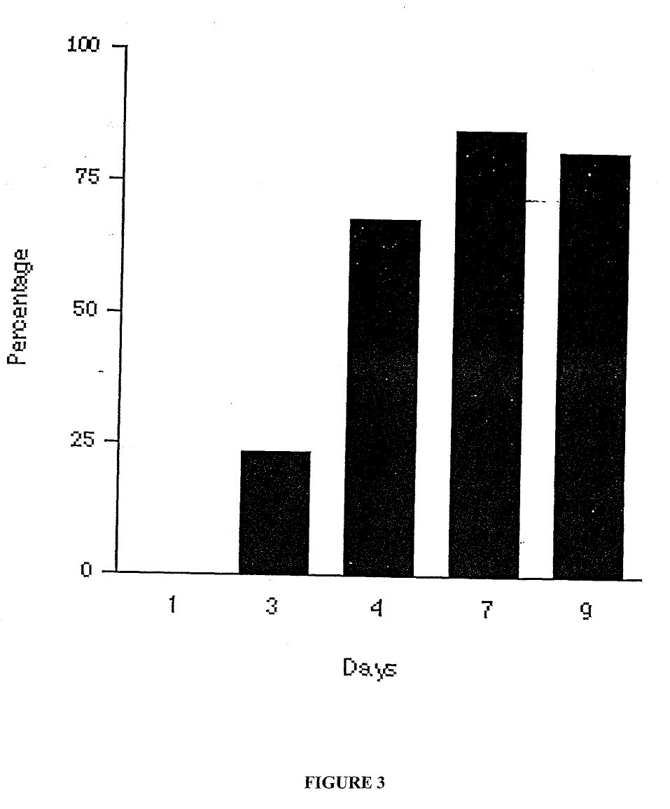

Presence of GFP, reflecting GFP expression, was monitored in vivo by ophthalmoscopy starting 24 hours after subretinal delivery of trans-lenti virus-GFP. GFP was first observed 3 days following injection in approximately 25% of injected eyes (data not shown). The percentage of eyes containing GFP increased rapidly, reaching a plateau of 80-85% on day 7. This number approximates 80%, the percentage of mouse eyes in which material is delivered accurately to the subretinal space (and doesn't leak into the vitreous; Bennett and Maguire, unpublished data). High levels of GFP persisted at least through day 111 (FIG. 3).

A numerical grading system ranging from 0 (no fluorescence) to 4 (maximal fluorescence) was used to quantitate the degree of GFP expression in each retina and to allow direct comparisons between different mouse eyes (see Methods section). In successfully transduced retinas, the intensity of GFP expression was 2 on day 3 (range: 1-3); 3 on days 4 through 111 (range 2-4) after subretinal injection of trans-lenti virus-GFP. The average number of eyes evaluated for each timepoint was 13 (except for the 111 day timepoint, where 4 were examined). All eyes in the last group expressed transgene, and half displayed an intensity grade of 4.

Histological examination of subretinally injected eyes revealed GFP in RPE cells, but not in photoreceptor cells. (Photoreceptors are centered in the GFP-negative outer nuclear layer (onl); data not shown). Onset of GFP expression was detected histologically at 48 hours after subretinal injection of trans-lenti virus-GFP (data not shown), 24 hours prior to detection by ophthalmoscopy. By day 7, the number of transgene-expressing RPE cells was significantly increased (data not shown). RPE cells continued to possess GFP more than 100 days after subretinal injection (n=4) (data not shown). Histological examination of surrounding tissues, including the optic nerve and optic chiasm, did not reveal transgene expression beyond the retina. Bright field microscopic examination of sections adjacent to transduced cells revealed no inflammatory cells, and immunohistochemical analysis confirmed the absence of CD4-, CD8-, and CD16-positive cells (data not shown).

B. Cell Mediated Responses to Trans-Lenti Virus—Specific Antigens and GFP Following Subretinal and Intramuscular Administration

Mice injected subretinally with trans-lenti virus-GFP were evaluated for DTH as an in vivo indicator of host cell-mediated responses. The animals were challenged intradermally with trans-lenti virus (intact virus, as described in methods) or GFP protein 7 days following subretinal priming; footpad thickness was measured 24 hours following footpad challenge, and compared to measurements prior to antigen challenge. No significant DTH response was observed upon challenge with lentiviral or GFP antigens in mice which received subretinal priming with trans-lenti virus-GFP. A control group of animals received injection of trans-lenti virus-GFP in skeletal muscles (gastrocnemius and tibialis anterior) and intradermal challenge 7 days later, as described above. Intramuscular injection of trans-lenti virus-GFP also failed to produce a DTH response. Tissue sections of gastrocnemius muscle from animals 7 days following intramuscular administration of trans-lenti virus-GFP confirmed that the viral vector had successfully transduced myocytes, as evidenced by GFP expression in these cells (data not shown).

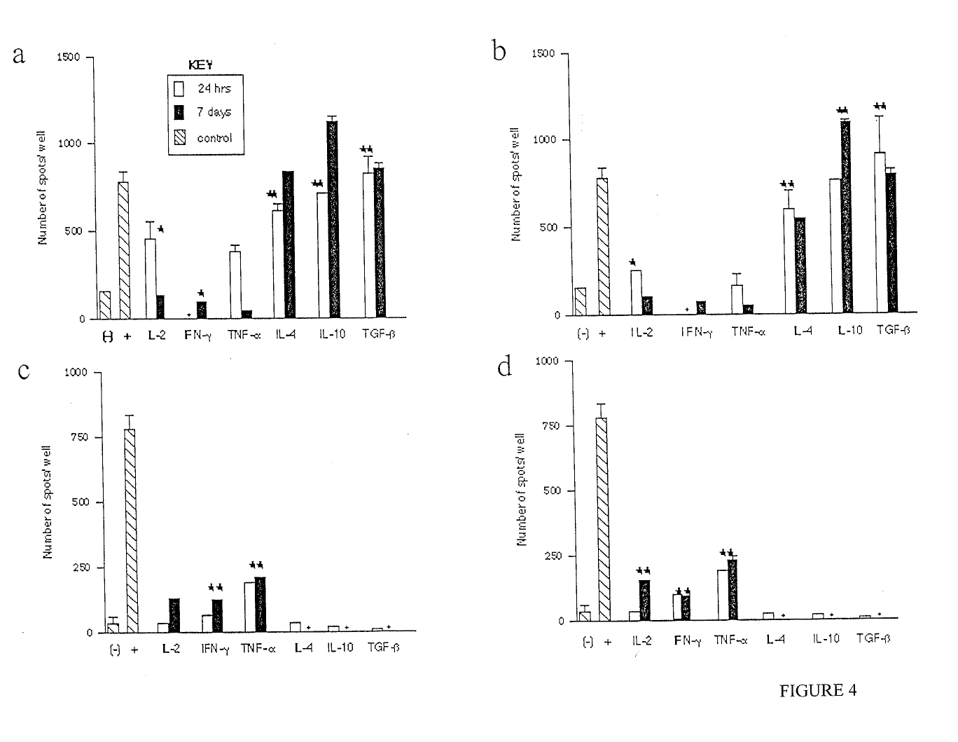

Cell mediated responses were analyzed in vitro using T-cell proliferation assays. At 24 hours and 7 days following subretinal injection of trans-lenti virus-GFP, splenic T-cells were isolated from mice after sacrifice. Elispot assays were used to detect the induction of cytokines specific to lentiviral and GFP antigens. Unstimulated cells from the same animals were used as controls. In the lentivirus-specific cytokine assay, all cytokine levels were elevated at 24 hrs after subretinal trans-lenti virus-GFP injection. At 7 days, spleen-derived T-cells produced primarily the immunosuppressive cytokines IL-4, IL-10, and TGF-β, while the pro-inflammatory cytokines IL-2, IFN-γ, and TNF-α were maintained close to baseline (unstimulated) levels (FIG. 4a). A similar cytokine profile was observed in the GFP-specific cytokine assay (FIG. 4b). Control animals receiving intramuscular injection of trans-lenti virus-GFP were also analyzed for cytokine induction at the corresponding time points by T-cell proliferation assay. In both the lentivirus- and GFP-specific cytokine assays, there was significant induction of TNF-α at 24 hours following intramuscular injection of trans-lenti virus-GFP (FIGS. 4c and 4d); however, levels of IL-2, IFN-γ, IL-4, IL-10, and TGF-β, were similar to those of unstimulated controls. At 7 days, levels of TNF-α remained elevated, and there was significant induction of IL-2 and IFN-γ, while levels of IL-4, IL-10, and TGF-β were close to baseline (FIGS. 4c and 4d).

C. Humoral Response to Subretinal and Intradermal Injection of Trans-Lenti Virus-GFP

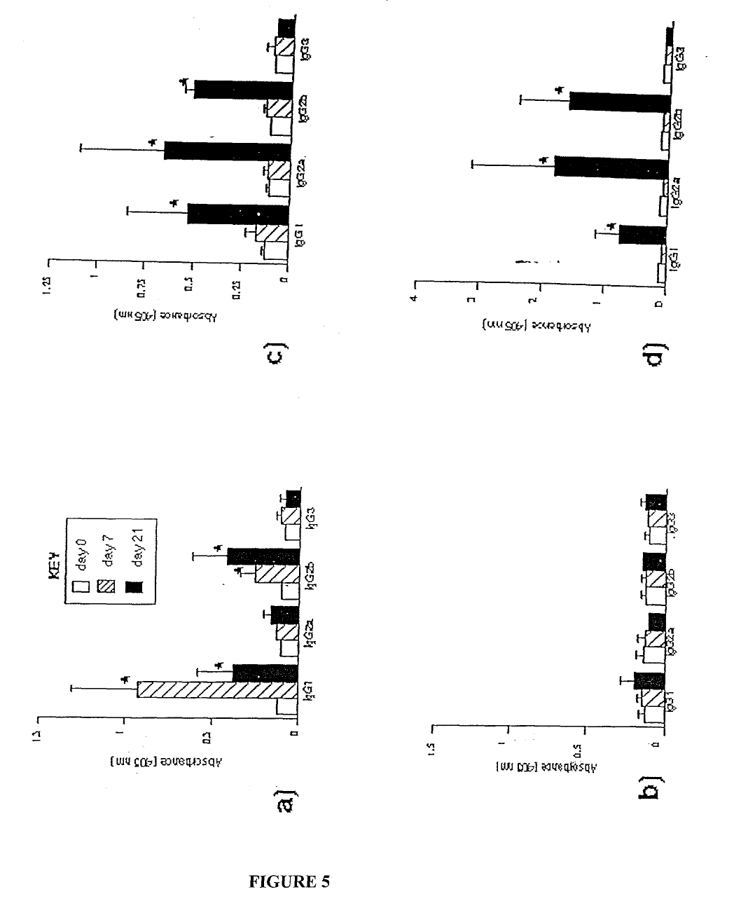

Serum samples from mice receiving subretinal and intramuscular injection of trans-lenti virus-GFP were collected on days 7 and 21, and analyzed for antibody isotypes by ELISA. Induction of a systemic humoral response was seen following both subretinal and intradermal administration of trans-lenti virus-GFP. Isotyping of the lentiviral-specific antibody response following subretinal injection revealed a predominant induction of IgG1 and IgG2b antibodies, while IgG2a and IgG3 levels remained near baseline (FIG. 5). The induction of these antibody isotypes was evident as early as one week following subretinal injection of trans-lenti virus-GFP, and IgG1 and IgG2b antibody levels remained elevated as late as 21 days following administration (FIG. 5a). Isotyping of the GFP-specific antibody response did not reveal significant induction of any antibody isotype at day 7 or 21 following subretinal injection of trans-lenti virus-GFP (FIG. 5b). Co-infection of human embryonic kidney epithelial cells with mixtures of trans-lenti virus-GFP and serum samples from subretinally-injected mice on days 7 and 21 resulted in 100% GFP expression at all dilutions tested, indicating that the lentivirus-specific antibodies induced by subretinal injection of trans-lenti virus-GFP were non-neutralizing in nature (data not shown).

Intramuscular administration of trans-lenti virus-GFP produced a distinct pattern of antigen-specific antibody isotypes. At 7 days following injection, no significant induction of lentivirus-specific antibodies was seen. However, by 21 days, however, there was induction of trans-lenti virus-specific IgG1, IgG2b, and IgG2a (FIG. 5c). Isotyping of the GFP-specific antibody response revealed a pattern similar to that of the trans-lenti virus-specific antibody response, with gradual induction of the antibody isotypes IgG1, IgG2b, and IgG2a by day 21 after intramuscular injection of trans-lenti virus-GFP (FIG. 5d). Intramuscular injection of trans-lenti virus-GFP failed to induce neutralizing antibodies (data not shown).

III. Discussion

The biological characteristics of retinal transduction using trans-lentivirus vector has been analyzed and the results presented here demonstrate a rapid initiation of transgene expression (within 2 days of exposure) lasting for more than 100 days after exposure. The cellular specificity, rapid onset, and stability of transgene expression make this vector an attractive candidate for treatment of both acute and chronic disease affecting the outer retina. One general concern relating to viral vectors is the fact that they can elicit strong cellular or humoral immune responses which result in elimination of the transgene, cellular toxicity, or an inability to readminister the vector. With lentiviral vectors, an additional reason to characterize the immune response is that the deleterious immunological effects of its parental virus, HIV, have been well documented. It is not known whether recombinant lentiviral vectors differ from the wildtype vectors in this response. This study characterized the nature of the host immune response elicited by subretinal administration of recombinant, replication-defective lentiviral vector. For these studies, the trans-lentiviral vector was selected which significantly reduces the likelihood of generation of replication-competent virus.

The results presented above demonstrate the cellular-specificity and duration of the trans-lenti mediated retinal transgene expression. Interestingly, the results also found that transgene expression is initiated soon after subretinal delivery, as early as 48 hours after injection. The most surprising finding, however, was the pattern of cytokine induction following subretinal injection of the vector. Animals injected subretinally developed significant induction of the Th2 cytokines 1L-4, IL-10, and TGF-β. Interestingly, there was a greater increase in IL-4 levels following in vitro lentiviral stimulation than with GFP (p=0.004). In contrast, systemic (intramuscular) injection of the vector induced the pro-inflammatory (Th1) cytokines IL-2, IFN-γ, and TNF-α. Similar patterns were observed for reaction to the GFP protein. The humoral response to subretinal lentiviral vector injection paralleled these findings. Following subretinal injection, both IgG1 and IgG2b antibody isotypes were induced, reflecting a Th2-type immune response. In addition, neutralizing antibodies were not formed, as judged by in vitro assays. In contrast, intramuscular injection of lentiviral vector induced significant levels of the antibody isotype IgG2a, which is characteristic of a Th1 immune response. As expected, given these results, inflammatory cells were not apparent in injected retinal tissue upon immunohistological examination.

Since the immunological responses differ (and are of the Th2 subtype) after subretinal versus systemic administration of the vector, we conclude that subretinal induces an immune deviant response. This is the first demonstration of an altered cytokine milieu as a result of administration of a viral antigen into the subretinal space. The data suggest that the subretinal space plays an active role in promoting an immune deviant response following the introduction of lentivirus. This process may be mediated by the RPE cells, which we have shown are the target cells of lentiviral vector transduction. RPE cells may serve as antigen presenting cells, processing and presenting viral antigen in an immune deviant fashion, perhaps under the influence of immunosuppressive cytokines, such as TGF-β. This particular cytokine is found in the interphotoreceptor matrix, which fills the subretinal space (Hewitt et al. (1994) Retina Basic Science and Inherited Retinal Disease, Vol. 1, Ogdon, T. E. ed. St. Louis, Mosby; 58-71.

Most of what is known about the immunological features of the eye derives from anterior chamber injection. The immune deviant response induced by injection of antigen into the anterior chamber of the eye, a phenomenon known as anterior chamber-associated immune deviation (ACAID), is characterized by suppression of DTH and the inability to produce complement-fixing antibodies (Streilein et al. (1987) FASEB J. 1: 199-208). Injection of soluble antigen protein into the anterior chamber can also result in an altered cytokine milieu, characterized by downregulation of the pro-inflammatory cytokine IFN-γ, and augmented levels of the immunosuppressive cytokines TGF-β, IL-4, and IL-10 (Kosiewicz et al.(1998) J. Immunol. 161:5382-5390).

### EXAMPLE 2

### Delivery of Trans-Lenti Virus-RPE65 into the Subretinal Space of RPE65−/− Mice

I. Introduction

A dog model of Leber congenital amaurosis (LCA) has proven useful in the study of the disease. This animal model, like the children it models, suffers from near total blindness in infancy. The dog model (like humans with the same disease) possesses homozygous mutations in the gene encoding RPE65. In the present study, we determine if the trans-lentivirus-mediated delivery of wildtype RPE65 could also result in rescue in the Rpe65^{-/-} mouse.

II. Materials and Methods

A. Vector preparation

Plasmids: The pPCW-RPE65 gene transfer vector was constructed by cloning a canine cDNA encoding wildtype RPE65 between the BamHI and XhoI restriction sites of pPCW. The wildtype canine RPE65 cDNA used in this experiment was PCR isolated previously. See, for example, Aguirre et al. (1998) Mol Vis. 4:23, herein incorporated by reference. Expression of the RPE65 gene from the gene transfer vector is controlled by the CMV promoter.

Preparation of vector stocks: The RPE65-containing trans-lenti vector stock (TLV-RPE65), was generated by transient co-transfection/expression of plasmid DNAs encoding the trans-lenti vector components in 293T cells. The 293T cells were seeded into forty-eight 6-well plates at 1×10⁶ cells per well. The following day, each well of subconfluent monolayer cultures of 293T cells were co-transfected with 3.0 μg of the pCMV-gag-pro packaging plasmid, 1.5 μg of the pCMV-vpr-RT-IN trans-enzyme plasmid, 2.0 μg of the pMD.G (VSVG) expression plasmid (env construct), and 3.0 μg of the pPCW-RPE65 gene transfer (vector) plasmid using calcium phosphate DNA precipitation. Sixty hours post-transfection, the 293T culture supernatants from all forty-eight 6-well plates were pooled, clarified by low-speed centrifugation (100 g, 10 min), and filtered through 0.45-μm pore-size filters. A concentrated (high-titer) vector stock was generated by pelleting the vector particles in an ultracentrifuge. The vector pellet was carefully resuspended in 1.0 ml of DMEM containing no serum. The trans-lenti virus-RPE65 stock was aliquoted, and stored at −80° C. Vector titers were approximated by determining the amount of capsid (p24 antigen) present within the trans-lenti virus-RPE65 stock using a commercially available ELISA kit for the detection of HIV-1 p24 (Coulter) and comparing them with the p24 levels measured in a GFP-containing trans-lenti vector stock of known titer. Using this method, the trans-lenti virus-RPE65 stock had a titer of 5×10⁸ IU/ml.

B. Animals

A breeding pair of Rpe65^{-/-} mice was generously provided by Michael Redmond. In preliminary experiments involving delivery of trans-lentivirus-RPE65, three adult Rpe65^{-/-} mice, aged 4.5 months, were studied.

C. Intraocular Administration of Trans-Lentivirus

Vector was administered unilaterally through subretinal injection, using a transscleral transchoroidal approach as previously described (Bennett et al. (1994) Investigative Ophthalmology & Visual Science 35: 2535-2542 and Liang et al. (2000) Intraocular delivery of recombinant virus. In Methods in Molecular Medicine: Ocular Molecular Biology Protocols. Edited by Rakoczy et al.: Humana Press Inc, 125-139, both of which are herein incorporated by reference. One microliter of virus-containing solution (containing a total of ˜5×10⁸ IU) was injected, creating a dome-shaped retinal detachment, which was visible under the microscope. The contralateral eyes received sham surgery, but no virus. Ophthalmoscopy was performed 1.5 months following injection in order to determine whether there were abnormalities present which would affect retinal function testing.

D. Retinal Function Testing

Pupillometry was performed 2.25 months after injection in order to determine whether there was communication between neurosensory retina and the central nervous system. Pupillometry was performed by exposing one pupil to light of increasing intensities and measuring papillary constriction with an infrared-illuminated camera attached to a video monitor. Recordings were performed on each eye, separately using an apparatus designed and built by Artur Cideciyan.

Electroretinograms were performed 2.5 months after injection in order to determine whether there was light-induced electrical response in the retina. Procedures were as described by Liang et al. (2000) Molecular Therapy 3: 241-248 and Liang et al. (2001) Molecular Therapy 3: 241-248, both of which are herein incorporated by reference. Briefly, bilateral simultaneous full-field electroretinograms (ERGs) were performed. ERGs were recorded using a custom-built ganzfeld, a computer-based system (EPIC-XL, LKC Technologies, Inc., Gaithersburg, Md.) and specially-made contact lens electrodes (Hansen Ophthalmic Development Lab, Iowa City, Iowa) (van Hooser et al. (2000) PNAS 97:8623-8628). Animals were dark-adapted for >12 hrs and anesthetized with intramuscular injections of ketamine HCl (60-75 mg/kg) and xylazine (5 mg/kg). Dark-adapted ERGs were elicited with blue flash stimuli (−0.3 log scot-cd.s.m⁻²); 2-10 responses were averaged with a 15 sec interstimulus interval. Flicker ERGs were recorded in the light-adapted state (>5 min) to a 15 Hz white stimulus (0.4 log cd.s.m⁻²); 50-100 responses were averaged. Dark-adapted ERGs were measured conventionally for b-wave amplitude and flicker ERGs for peak-to-peak amplitude.

E. Tissue Studies

Following retinal function testing, animals were euthanized. Prior to enucleation, eyes were marked by cautery at the limbus in the temporal quadrant. This mark served as a reference point for orientation of the eye during embedding. The eyes were enucleated and fixed in 4% paraformaldehyde in phosphate-buffered saline (PBS). Eyes were cryo-protected in 30% sucrose made in PBS and then embedded and frozen in Tissue-Tek freezing compound. Tissue was serially sectioned at 15 um. Every 20^{th} section was used for immunofluorescence in order to assess the presence of RPE65 protein. Immunofluorescence was performed using a rabbit polyclonal antibody directed against RPE65 and an FITC-labeled secondary anti-rabbit antibody. Sections were viewed on a Leica DM microscope (Leica Microsystems Inc., Wetzlar, Germany) under epifluorescence illumination.

III. Results

A. Ophthalmoscopy

In all three mice (animal numbers AVRP15-2, AVRP16-2 and AVRP17-2) and in all six eyes) media was clear and visualization of the retina was successful. There was no inflammation and the retinas were flat. The injection sites could be visualized in ⅔ of the trans-lenti viral-RPE65-injected eyes (in animals AVRP16-2 and AVRP17-2).

B. Pupillometry

Two of the mice (AVRP16-2, AVRP17-2) had asymmetric pupillometry results suggesting that the trans-lenti viral-RPE65-treated eyes had greater retinal/CNS function than the control eyes. There were no significant differences in pupillary response in the third animal.

C. Electroretinograms (ERGs)

Animal AVRP17-2 died of anesthesia complications during ERG recording. Animal AVRP16-2 had remarkably larger ERG amplitudes (a wave and b wave) in the trans-lenti viral-RPE65-treated eye than in the control eye. There were also lower light thresholds identified in this eye compared to the control eye.

D. Immunofluorescence

The trans-lentiviral-RPE65-treated eye of animal AVRP16-2 possessed high levels of RPE65 protein in retinal pigment epithelium (RPE) cells only. RPE65 protein was detected over a significant proportion of the eye (˜45%). A small amount of RPE65 protein was identified around the injection site in the trans-lentiviral-RPE65-treated eye of AVRP17-2. There was no detectable RPE65 protein in control eyes.

IV. Discussion

The data presented above suggests that subretinal delivery of trans-lenti viral-RPE65 ameliorates the phenotype in Rpe65^{-/-} mice. Two out of three animals injected with trans-lenti viral-RPE65 showed asymmetric pupillary responses and at least one of these two animals (one died of anesthesia) revealed significantly improved ERG responses in the treated eye. In that eye, there was evidence of a significant amount of RPE65 protein in RPE cells (in nearly 50% of the area). A smaller amount of RPE65 protein was detected in the animal with weaker pupillometry responses (animal AVRP17-2). The results suggest that the amount of RPE65 protein is correlated with the extent of rescue of visual function.

Additional Rpe65^{-/-} animals have since received subretinal injection of TLV-RPE65 and the visual function studies are pending. Both neonatal and adult animals have been treated unilaterally and contralateral eyes have been injected with saline as control or sham-injected.

The present inventions has been described more fully hereinabove with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. A method for delivering a nucleotide sequence of interest to a retinal cell of a mammal comprising administering to said retinal cell a trans-viral vector particle having a nucleotide sequence of interest operably linked to a promoter active in said retinal cell, wherein said nucleotide sequence of interest has retinal therapeutic properties.

2. The method of claim 1, wherein said trans-viral vector is a trans-lentiviral vector.

3. The method of claim 1, wherein said trans-viral vector is a trans-retroviral vector.

4. The method of claim 1, wherein said retinal cell is a retinal pigment epithelium (RPE) cell.

5. The method of claim 1, wherein said nucleotide sequence having retinal therapeutic properties encodes an antisense nucleotide sequence, a ribozyme, or a polypeptide.

6. The method of claim 5, wherein said polypeptide comprises a growth factor.

7. The method of claim 5, wherein said polypeptide comprises RPE65 or a biologically active fragment or variant thereof.

8. The method of claim 7, wherein said polypeptide comprises RPE65.

9. The method of claim 1, wherein said trans-viral vector particle is administered to a mammalian retinal cell cultured in vitro.

10. The method of claim 9, wherein said trans-viral particle is a trans-lentiviral particle.

11. The method of claim 9, wherein said trans-viral particle is a trans-retroviral particle.

12. The method of claim 1, wherein the trans-viral vector particle is administered at a concentration of 4×10³ to 4×10⁷ infectious units.

13. A method of treating a retinal disorder comprising administering to a retinal cell of a mammal in need thereof, a therapeutically effective concentration of a trans-viral vector particle, wherein said trans-viral vector particle comprises a proviral genome having a nucleotide sequence of interest operably linked to a promoter active in said retinal cell, wherein said nucleotide sequence has retinal therapeutic properties.

14. The method of claim 13, wherein said trans-viral particle is a trans-lentiviral particle.

15. The method of claim 13, wherein said trans-viral particle is a trans-retroviral particle.

16. The method of claim 13, wherein said nucleotide sequence having retinal therapeutic properties encodes an antisense nucleotide sequence, a ribozyme, or a polypeptide.

17. The method of claim 13, wherein said trans-viral vector particle is administered to the retinal cell via subretinal injection.

18. The method of claim 13, wherein said retinal cell is a retinal pigment epithelium (RPE) cell.

19. The method of claim 13, wherein said retinal disorder is a retinal degeneration disorder.

20. The method of claim 13, wherein said retinal degeneration disorder is selected from the group consisting of retinitis pigmentosa and macular degenerative diseases.

21. The method of claim 19, wherein said retinal degeneration disorder is Leber congenital amaurosis.

22. The method of claim 21, wherein said nucleotide sequence of interest encodes RPE65 or a biologically active fragment or variant thereof.

23. The method of claim 22, wherein said nucleotide sequence of interest encodes RPE65.

24. The method of claim 23, wherein the trans-viral vector particle is administered at a concentration of 4×10³ to 4×10⁷ infectious units.

25. A packaging cell line having an env construct, a packaging construct, a trans-enzyme construct, and a gene transfer vector wherein said gene transfer vector comprises a nucleotide sequence of interest operably linked to a promoter active in a target cell, wherein said nucleotide sequence of interest encodes RPE65 or a biologically active variant or fragment thereof.

26. The method of claim 25, wherein said nucleotide sequence of interest encodes RPE65.

27. A trans-viral vector particle comprising a proviral genome having a nucleotide sequence of interest operably linked to a promoter active in a target cell, wherein said nucleotide sequence of interest encodes RPE65 or a biologically active variant or fragment thereof.

28. The trans-viral vector particle of claim 27, wherein said nucleotide sequence of interest encodes RPE65.

29. The trans-viral vector particle of claim 27, wherein said target cell is a retinal cell.

30. A pharmaceutical composition comprising a trans-viral vector particle, wherein said trans-viral vector particle comprises a proviral genome having a nucleotide sequence of interest operably linked to a promoter active in a target cell, wherein said nucleotide sequence encodes RPE65 or a biologically active fragment or variant thereof.

31. The pharmaceutical composition of claim 30, wherein said nucleotide sequence of interest encodes RPE65.

32. A method for generating a trans-viral vector particle comprising:
a) providing a packaging cell having an env construct, a packaging construct, and a trans-enzyme construct;
b) introducing into said packaging cell a gene transfer vector comprising a nucleotide sequence of interest operably linked to a promoter active in a target cell, wherein said nucleotide sequence of interest encodes RPE65 or a biologically active fragment or variant thereof; and,
c) incubating the packaging cell of step (b) under conditions wherein the trans-viral vector particle is produced.
